(19) United States
(12) Patent Application Publication
(10) Pub. No.: US 2009/0047279 A1
(43) Pub. Date: Feb. 19, 2009

(54) (R)-N-Stereoisomers of 7,8-Saturated-4,5-Epoxy-Morphinanium Analogs

(75) Inventors: Julio Perez, Tarrytown, NY (US); Amy Qi Han, Hockessin, DE (US); Yakov Rotshteyn, Monroe, NY (US)

Correspondence Address: KELLEY DRYE & WARREN LLP, 400 ALTLANTIC STREET , 13TH FLOOR, STAMFORD, CT, 06901, US

(73) Assignee: Progenics Pharmaceuticals, Inc., Tarrytown, NY (US)

(21) Appl. No.: 11/944,389

(22) Filed: Nov. 21, 2007

Related U.S. Application Data
(60) Provisional application No. 60/867,099, filed on Nov. 22, 2006.
(60) Provisional application No. 60/867,390, filed on Nov. 27, 2006.

(51) Int. Cl. A61K 39/395; C07D 491/08; A61K 31/439; A61P 1/10; A61P 1/00; A61K 36/00

(52) U.S. Cl. 424/133.1; 546/45; 514/282; 424/725

(57) ABSTRACT

Novel (R)—N-stereoisomers of 7,8-saturated-4,5-epoxy-morphinanium analogs are disclosed. Pharmaceutical compositions containing the (R)—N-stereoisomers of 7,8-saturated-4,5-epoxy-morphinanium analogs and methods for their pharmaceutical uses are also disclosed. Such analogs are disclosed as being useful in treating, among varying conditions, opioid-induced constipation.

### BACKGROUND OF THE INVENTION

1. Field of the Invention

The present invention generally relates to (R)—N-stereoisomers of 7,8-single-bond-4,5-epoxy-morphinanium analogs (hereinafter referenced to as “7,8-saturated-4,5-epoxy-morphinaniums”), including 7,8-dihydro-4,5-epoxy-morphinanium analogs, synthetic methods for their preparation, pharmaceutical preparations comprising the same, and methods for their use. This application claims priority to U.S. application 60/867,099, filed Nov. 22, 2006, and to U.S. application 60/867,390, filed Nov. 27, 2006, and hereby incorporates each in entirety.

2. Description of the Related Art

The medicinal and psychological effects of opium have been known since ancient times. It was not, however, until around the beginning of the nineteenth century, that morphine was isolated from opium, and codeine and papaverine thereafter. By the middle of the nineteenth century pure alkaloids rather than crude opium preparations were becoming established medical practice. Since the nineteenth century a host of synthetic and semi-synthetic derivatives of these natural alkaloids have been made.

In respect of morphinan compounds, it is now known that substituent substitutions can have significant effects on the pharmacology. For example, some have reported that the 3-hydroxy morphinans may be significantly less effective orally than parenterally possibly due to a significant first-pass metabolism. Glucuronidation of morphine at its 3-hydroxyl group is believed to terminate the activity. However, 3-methoxy groups such as seen in oxycodone and codeine have been associated by some with good oral potency.

Opioid activity of morphinoids has been shown to be particularly sensitive to the nature of their nitrogen substituents. For example, replacement of the N-methyl group in morphine and related opioids by substituents rich in π-electrons, such as allyl, cylcobutylmethyl, and propylmethyl, result in potent antagonists such as nalorphine, naloxone, naltrexone and nalbuphine.

The designations “R” and “S” are commonly used in organic chemistry to denote specific configuration of a chiral center. The designations “R” refers to “right” and refers to that configuration of a chiral center with a clockwise relationship of group priorities (highest to second lowest) when viewed along the bond toward the lowest priority group. The term “S” or “left” refers to that configuration of chiral center with a along the bond toward the lowest priority group.

The priority of groups for the R/S designation is based upon atomic number (heaviest isotope first). A partial list of priorities and a discussion of stereochemistry is contained in the book: The Vocabulary of Organic Chemistry, Orchin, et al. John Wiley and Sons, Inc., page 126 (1980), which is incorporated herein by reference in its entirety. When quaternary nitrogen morphinan structures are produced, such structures may be characterized as (R) or (S) stereoisomers.

The art suggests that isolated stereoisomers of a compound, whether enantiomers or diasteromers, sometimes may have contrasting physical and functional properties, although it is unpredictable whether this is the case in any particular circumstance. Dextromethorphan is a cough suppressant, whereas its enantiomer, levomethorphan, is a potent narcotic. (R,R)-methylphenidate is a drug to treat attention deficit hyperactivity disorder (ADHD), whereas its enantiomer, (S,S)-methylphenidate is an antidepressant. (S)-fluoxetine is active against migraine, whereas its enantiomer, (R)-fluoxetine is used to treat depression. The (S)-enantiomer of citalopram is therapeutically active isomer for treatment of depression. The (R)-enantiomer is inactive. The (S)-enantiomer of omeprazole is more potent for the treatment of heartburn than the (R) enantiomer.

Caldwell et al., Complete Proton and Carbon Nuclear Magnetic Resonance Spectral Assignments of Some Morphin-6-one Alkaloids by Two-Dimensional NMR Techniques, describe the use of two-dimensional NMR conformational analysis Nuclear Overhauser enhancement difference analysis) with respect to select quaternary N-methyl oxycodone analogs to determine that the N-methyl group was in the equatorial position. They noted that proton coupling constants with respect to the compounds they tested suggested that the cyclohexanone ring and piperidine rings of the morphinan backbone adopt slightly distorted chair conformations.

Bianchetti et al., Quaternary Derivatives of Narcotic Antagoists: Sterochemical Requirements at the Chiral Nitrogen for In Vitro and In Vivo Activity, 1983 Life Science 33 (Sup I):415-418 studied three pairs of diastereoisomers of quaternary narcotic antagonist and their parent tertiary amines, levallorphan, nalorphine, and naloxone, to see how the configuration about the chiral nitrogen affected in vitro and in vivo activity. It was found that the activity varied considerably depending on how the quaternary derivatives were prepared. In each series, only the diastereomer obtained by methylation of the N-allyl-substituted tertiary amine (referred to as “N-methyl diastereomer”) was potent in displacing ³H-naltrexone from rat brain membranes, and acting as a morphine antagonist in the guinea-pig ileum. Conversely, diastereoisomers obtained by reacting N-methyl-substituted tertiary amines with allyl halide (referred to as “N-allyl diastereomers”) did not displace 3H-naltrexone and had negligible antagonist activity and slight agonist action in the guinea-pig ileum. In vivo findings were generally consistent with those in vitro. Thus only the “N-methyl” but not the “N-allyl diastereomers” inhibited morphine-induced constipation in rats and behaved as antagonists. The author stated that the prepared materials appeared to be pure by ¹H and ¹³C nuclear magnetic resonance (NMR) analysis, but these methods are not accurate. The author cites a literature reference for the assignment of the (R) configuration to the “N-methyl diastereomer” of nalorphine. No assignment is proposed for the levallorphan and naloxone diastereomers. It would be adventurous to extrapolate the configuration to these diastereomers (R. J. Kobylecki et al, J. Med. Chem. 25, 1278-1280, 1982).

Kobylecki et al., 1982, N-Methylnalorphine: Definition of N-allyl conformation for antagonism at the opiate receptor, J. Med. Chem. 25:1278-1280 report, based on X-ray diffraction data, that the active diastereomer derived from nalorphine (N-methyl diasteromer) the allyl group about the quarternary nitrogen has an equatorial configuration. Kobylecki reported that the isomer with the axial N-substituent demonstrated some agonist activity (although very low) with very substantial antagonist activity in comparison (to its agonist activity) whereas the equatorial N-substituent displayed pure opioid antagonist activity.

Iorio et al., Narcotic angonist/antagonist properties of quaternary diasteromers derived from oxymorphone and naloxone, 1984, Chim. Ther. 19: 301-303, indicates that correlations between agonist and antagonist ratio and N-substitution orientation follow the same pattern found by Kobylecki with respect to diasteroisomeric quaternary morphinanium salts, that is, that compounds with larger groups equatorially displayed more antagonist activity than the corresponding axial diastereoisomer. These authors suggest that all types of activity, agonism, antagonism and mixed activity, may all be explained by different conformational types of interaction of equatorial N-substituents with receptor subsites. Comparison of activity of the compounds they produced was by direct in vitro ileum contraction tests, and in vivo by injecting the compounds into the brain of mice. Funke and deGraaf, A ¹H and ¹³C nuclear magnetic resonance study of three quaternary salts of naloxone and oxymorphone, 1986, J. Chem. Soc. Perkin Trans. II 735-738, referencing Ioria et al., report the ¹H and ¹³C n.m.r. data with three N,N-dialkyl-morphinanium chloride derivates (one N,N-diallyl and two N-allyl-N-methyl diastereoisomers).

Cooper (U.S. Pat. No. 6,455,537) disputes the relevancy of the Iorio in vivo data arguing that the administration into the brain was not appropriate given that quaternized agents do not pass into the brain. Cooper performing a number of in vivo tests using intravenous methylnalorphine, found that the (R)-isomer of N-methylnalorphine provided superior treatment to antagonize or prevent opiate induced side effects in mammals such as nausea, vomiting and ataxia, when compared with the (S)-isomer or a mixture of R/S N-methylnalorphine.

Feinberg et al., The opiate receptor: A model explaining structure-activity relationships of opiate agonists and antagonists, 1976 Proc. Natl. Acad. Sci. USA 73: 4215-4219, opine that the spatial location of “antagonist substituents” such as N-allyl and cyclopropylmethyl, determine the “purity” of the antagonistic pharmacological properties of an opioid drug. Feinberg et al. theorize that a 14-hydroxyl group on the morphinan structure helps to increase the proportion of antagonistic substituents in the equatorial conformation relative to axial conformation in respect of the piperidine ring, and that such equatorial confirmation at least with respect to N-allyl and cyclopropylmethyl increase the “pure” antagonism. They further theorize that in mediating antagonist activity that the specific antagonist binding site of the receptor interacts with the pi-electrons of the N-allyl or the atomic configurations to N-cyclopropylmethyl or N-cyclobutylmethyl groups, which are required for antagonist pharmacology, thus stabilizing antagonist receptor conformation. To secure “pure” antagonist properties, they suggest that the approximation of the antagonist substituent to the antagonist binding site of the receptor must be facilitated by a 14-hydroxyl or 9-β-methyl substituent as seen in naloxone or benzomorphan antagonists. Without such substituents, they hypothesize varying mixtures of agonist and antagonist pharmacology.

While such references may suggest improved antagonistic activity for certain functional groups on a morphinan nitrogen when such groups an equatorial position, in conjunction they do not suggest the agonist-antagonist activity of isolated (R)/(S) conformers or axial-equatorial conformers for morphinan compounds with different substituents, particularly with respect to compounds supporting different saturation profiles in respect of the rings of the backbone morphinan structure, compounds carrying a quaternary charged nitrogen, and compounds with different substituent pairs at the 3 and 6 positions of the morphinan backbone.

### SUMMARY OF THE INVENTION

Disclosed in embodiments described herein are (R)-7,8-saturated-4,5-epoxy-morphinanium analogs which have been produced in high purity, permitting the characterization of their relative retention time in chromatography versus that of their corresponding (S)-dihydro-4,5-epoxy-morphinanium stereoisomer. The isolated diastereomers of such analogs have been found to have activity different from that of their corresponding diastereomeric mixtures when the moieties attached to the nitrogen are C₁-C₈ alkyls or C₁-C₆ alkyls.

In an embodiment of the present invention, there is provided substantially or highly pure (R)-7,8-saturated-4,5-epoxy-morphinaniums, crystals of substantially of highly pure (R)-7,8-saturated-4,5-epoxy-morphinaniums and intermediates thereof, novel methods for making substantially or highly pure (R)-7,8-saturated-4,5-epoxy-morphinanium compounds, methods for analyzing, quantitating and isolating (R)-7,8-saturated-4,5-epoxy-morphinanium compounds in a mixture containing counterpart (S)-7,8-saturated-4,5-epoxy-morphinanium stereoisomer and its (R)-7,8-saturated-4,5-epoxy-morphinanium stereoisomer, methods of distinguishing an (S)-7,8-saturated-4,5-epoxy-morphinanium from its (R)-7,8-saturated-4,5-epoxy-morphinanium counterpart, pharmaceutical products containing the same and related uses of these materials.

Salts of (R)-7,8-saturated-4,5-epoxy-morphinaniums are also provided. A protocol for obtaining (R)-7,8-saturated-4,5-epoxy-morphinaniums is also provided. In addition, it has been discovered, surprisingly, that (R)-7,8-saturated-4,5-epoxy-morphinaniums of the present disclosure have opioid antagonist activity. The invention provides synthetic routes for stereoselective synthesis of these (R)-7,8-saturated-4,5-epoxy-morphinaniums, substantially pure (R)-7,8-saturated-4,5-epoxy-morphinaniums, crystals of substantially pure (R)-7,8-saturated-4,5-epoxy-morphinaniums, pharmaceutical preparations containing substantially one or more pure (R)-7,8-saturated-4,5-epoxy-morphinaniums, and methods for their use.

According to one embodiment of the invention, a composition is provided that comprises a 7,8-saturated-4,5-epoxy-morphinanium in the (R) configuration (that is, with respect to the nitrogen) is present at greater than 99.5%. In other embodiments the 7,8-saturated-4,5-epoxy-morphinanium in (R)-configuration (with respect to the nitrogen) is present in the composition in greater than about 99.6%, or about 99.7%, or about 99.8%, or about 99.9%, or about 99.95%, or greater than 99.95%. In one embodiment, there is no detectable counterpart (S)-7,8-saturated-4,5-epoxy-morphinanium compound in the analyzed composition using the chromatographic procedures described herein. It may be preferred that the composition is free of the corresponding (S)-7,8-saturated-4,5-epoxy-morphinanium as detected on HPLC. In one embodiment, there is no HPLC detectable counterpart (S)-7,8-saturated-4,5-epoxy-morphinanium at a detection limit of 0.02% and a quantitation limit of 0.05%. In yet another embodiment the composition of the invention contains 99.85% of the 7,8-saturated-4,5-epoxy-morphinanium in the (R)-configuration with respect to nitrogen, and it contains the counterpart stereoisomeric (S)-7,8-saturated-4,5-epoxy-morphinanium compound at a HPLC detectable detection limit of 0.02% and a quantitation limit of 0.05%.

According to one aspect of the invention, a composition is provided that comprises a 7,8-saturated-4,5-epoxy-morphinanium, wherein at least 99.6%, 99.7%, 99.8%, 99.85%, 99.9%, and even 99.95% of the 7,8-saturated-4,5-epoxy-morphinanium compound in the composition is in the (R)-configuration with respect to nitrogen, and the composition includes one or more of: a buffering agent, a chelating agent, a preserving agent, a cryoprotecting agent, a lubricating agent, a preservative, an anti-oxidant, or a binding agent.

According to one aspect of the invention, a composition is provided. The composition is an isolated compound of the (R) configuration with respect to nitrogen of Formula Z:

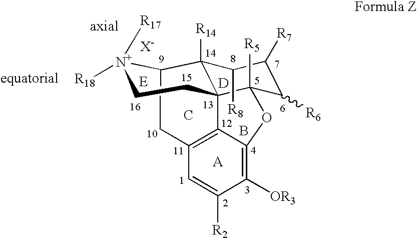

((R)-7,8-saturated-4,5-epoxy-morphinanium)
   wherein X is a counterion and R₁₇ and R₁₈ are selected to result in an (R) configuration about the nitrogen in conformity with the Cahn, Ingold, Prelog configuration assignment rules. R₃ may be a hydroxyl protecting group. The hydroxyl protecting group can be any of numerous such groups. In embodiments it is selected from the group consisting of: isobutyryl, 2-methyl butyryl, tertbutyl carbonyl, silyl ethers, 2-tetrahydropyranyl ethers, and alkyl carbonates. In one embodiment, the hydroxyl protecting group is isobutyryl.

(R)-7,8-saturated-4,5-epoxy-morphinaniums, as illustrated, are salts. Therefore, there will be a counterion, which for the present application includes the zwitterion. More typically, the counterion may be a halide, sulfate, phosphate, nitrate, or anionic-charged organic species. Halides include fluoride, chloride, iodide and bromide. In some embodiments, the halide is iodide and in other embodiments, the halide is bromide. In some embodiments the anionic-charged species is a sulfonate or a carboxylate. Examples of sulfonates include mesylate, besylate, tosylate, and triflate. Examples of carboxylates include formate, acetate, citrate, and fumarate.

According to another aspect of the invention, the foregoing compositions that comprise in a (R)-configuration with respect to nitrogen in some embodiments is a crystal, a solution, or a bromide salt of a 7,8-saturated-4,5-epoxy-morphinanium. In other embodiments, the foregoing compositions are pharmaceutical preparations, preferably in effective amounts and with a pharmaceutically acceptable carrier.

According to one aspect of the invention, a crystal of a certain 7,8-saturated-4,5-epoxy-morphinanium is provided that is at least about 99.5%, or about 99.6% or about 99.7%, or is about 99.8%, or about 99.9%, or greater than 99.95% of the 7,8-saturated-4,5-epoxy-morphinanium in (R)-configuration with respect to the nitrogen.

According to another embodiment of the invention, an (R)-7,8-saturated-4,5-epoxy-morphinanium compound is provided in isolated form. By isolated, it is meant at least 50% pure. In embodiments, the (R)-7,8-saturated-4,5-epoxy-morphinanium is provided at 75% purity, at 90% purity, at 95% purity, at 98% purity, and even at 99% purity or 99.5% versus the (S) form. In an embodiment, the (R)-7,8-saturated-4,5-epoxy-morphinanium is in a crystal form.

According to another aspect of the invention, a composition is provided. The composition comprises a 7,8-saturated-4,5-epoxy-morphinanium, wherein the 7,8-saturated-4,5-epoxy-morphinanium present in the composition is greater than 10% in (R) configuration with respect to nitrogen. More preferably, the 7,8-saturated-4,5-epoxy-morphinanium present in the composition is greater than 30%, 40%, 50%, 60%, 70%, 75%, 80%, 85%, 90%, 95%, 96%, 97%, 98%, 98.5%, 99%, 99.5%, 99.6%, 99.7%, 99.8%, and even 99.9% in (R) configuration with respect to nitrogen. In some embodiments there is no detectable counterpart (S)-7,8-saturated-4,5-epoxy-morphinanium compound as measured by high performance liquid chromatography (HPLC).

The composition in some embodiments is a solution, in others an oil, in others a cream, and in still others a solid or semi-solid. In one embodiment, the composition is a crystal.

According to another aspect of the invention, a pharmaceutical preparation is provided. The pharmaceutical preparation includes any one of the compositions of a particular (R)-7,8-saturated-4,5-epoxy-morphinanium described above in a pharmaceutically acceptable carrier. The pharmaceutical preparation contains a effective amount of the (R)-7,8-saturated-4,5-epoxy-morphinanium. In some embodiments, there is little or no detectable counterpart (S)-7,8-saturated-4,5-epoxy-morphinanium structure in the composition. If present, (R)-7,8-saturated-4,5-epoxy-morphinanium compound is at a level such that effective amounts of the (R)-7,8-saturated-4,5-epoxy-morphinanium compound are administered to a subject. In some embodiments, the pharmaceutical preparation further includes a therapeutic agent other than the 7,8-saturated-4,5-epoxy-morphinanium. In one embodiment, the therapeutic agent is an opioid or opioid agonist. Examples of opioids or opioid agonists are alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucuronide, nalbuphine, nalorphine, opium, oxycodone, oxymorphone, pentazocine, propiram, propoxyphene, remifentanyl, sufentanil, tilidine, trimebutine, tramadol, or combinations thereof. In some embodiments, the opioid or opioid agonist does not readily cross the blood brain barrier and, therefore, has substantially no central nervous system (CNS) activity when administered systemically (i.e., it is of the class of agents known as “peripherally acting”) agents. In one embodiment, the peripheral opioid agonist is a (S)-7,8-saturated-4,5-epoxy-morphinanium. In other embodiments, the therapeutic agent is not an opioid, opioid agonist, or an opioid antagonist. For example, the therapeutic agent can be a non-opioid analgesic/anti-pyretic, an antiviral agent, antibiotic agent, antifungal agent, antibacterial agent, antiseptic agent, anti-protozoal agent, anti-parasitic agent, anti-inflammatory agent, a vasoconstrictor agent, a local anesthetic agent, an anti-diarrheal agent, an anti-hyperalgesia agent, or combinations thereof.

In other embodiments the therapeutic agent is an opioid antagonist. Opioid antagonists include peripheral mu opioid antagonists. Examples of peripheral mu opioid antagonists include quarternary derivatives of noroxymorphone (See Goldberg et al, U.S. Pat. No. 4,176,186, and Cantrell et al WO 2004/043964), piperidine N-alkylcarboxylates such as described in U.S. Pat. Nos. 5,250,542; 5,434,171; 5,159,081; 5,270,328; and 6,469,030, opium alkaloid derivatives such as described in U.S. Pat. Nos. 4,730,048; 4,806,556; and 6,469,030, quaternary benzomorphan compounds such as described in U.S. Pat. Nos. 3,723,440 and 6,469,030.

In one embodiment of the invention, the (R)-7,8-saturated-4,5-epoxy-morphinanium is combined with an anti-diarrhea agent that is loperamide, loperamide analogs, N-oxides of loperamide and analogs, metabolites and prodrugs thereof, diphenoxylate, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, kaolin, pectin, activated charcoal, sulphaguanidine, succinylsulphathiazole, phthalylsulphathiazole, bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines, plant-derived anti-diarrheal agents or combinations thereof.

According to another embodiment, a method is provided for stereoselective synthesis of a 3-O-protected (R)-7,8-saturated-4,5-epoxy-morphinanium salt comprising methylating a 3-O-protected-appropriate morphinan compounds with a methylating agent to yield the desired 3-O-protected-(R)-(R)-7,8-saturated-4,5-epoxy-morphinanium salt. The hydroxyl protecting group of the 3-O-protected group in certain embodiments is isobutyryl, 2-methyl butyryl, tertbutyl carbonyl, silyl ethers, 2-tetrahydropyranyl ethers, and alkyl carbonates. The 3-O-protected (R)-compound may be a salt with an anion that can be, for example, a halide, sulfate, phosphate, nitrate or an organic anionic-charged species. The halide may be bromide, iodide, chloride, or fluoride. The organic anionic-charged species can be, for example, a sulfonate or carboxylate. Exemplary sulfonates are mesylate, besylate, tosylate, or triflate. Exemplary carboxylates are formate, acetate, citrate, or fumarate. The method can further involve exchanging the anion with a different anion. The alkylating agent can be an alkyl group susceptible to nucleophilic attack, and a leaving group. Exemplary methylating agents may be selected from the group consisting of methyl halide, dimethyl sulfate, methyl nitrate and methyl sulfonate. Methyl halides are methyl iodide, methyl bromide, methyl chloride and methyl fluoride. Methyl sulfonates include methyl mesylate, methyl besylate, methyl tosylate, and methyl triflate. In one embodiment, the alkylation is conducted at a temperature range from about >70° C. to about 100° C., or from 80° C. to about 90° C., or at about 88° C. The alkylation reaction may be conducted for a significant period of time, for example, about 1 hour to 24 hours, or about 5 hour to 16 hours or for about 10 hours. The method can further involve purification of the 3-O-protected (R)-7,8-saturated-4,5-epoxy-morphinanium salt using at least one purification technique, such as chromatography or recrystallization. The chromatography can be reverse-phase chromatography or regular phase chromatography. In some embodiments, the regular phase chromatography can use alumina or silica gel. The 3-O-protected-intermediate can be purified prior to alkylation.

According to another aspect of the invention a method for isolation and purification of (R)-7,8-saturated-4,5-epoxy-morphinaniums is provided, comprising passing the crude (R)-7,8-saturated-4,5-epoxy-morphinaniums through a chromatography column and collecting the (R)-7,8-saturated-4,5-epoxy-morphinaniums which elutes at the (R)-7,8-saturated-4,5-epoxy-morphinaniums retention time. This process can be in addition to the method described above, after the deprotecting step and/or the anion exchange resin column step.

According to another aspect of the invention a method for analyzing (R)-7,8-saturated-4,5-epoxy-morphinaniums in a mixture of (R)-7,8-saturated-4,5-epoxy-morphinaniums and (S)-7,8-saturated-4,5-epoxy-morphinaniums is provided. The method involves conducting high performance liquid chromatography (HPLC) and applying (R)-7,8-saturated-4,5-epoxy-morphinaniums to the chromatography column as a standard. The method preferably involves applying both (R)-7,8-saturated-4,5-epoxy-morphinaniums and (S)-7,8-saturated-4,5-epoxy-morphinaniums as standards to determine relative retention/elution times. Relative retention times of (R) and (S) are described herein.

In one embodiment, the chromatography is conducted using two solvents, solvent A and solvent B, wherein solvent A is an aqueous solvent and solvent B is a methanolic solvent and wherein both A and B contain trifluoroacetic acid (TFA). A may be 0.1% aqueous TFA and B is 0.1% methanolic TFA. In embodiments the column comprises a bonded, end-capped silica. In embodiments, the pore size of the column gel is 5 microns. In an embodiment, the column, flow rate and gradient program are as follows:

| | |
|---|---|
| Column: | Luna C18(2), 150 × 4.6 mm, 5 μ |
| Flow Rate: | 1 mL/min |

### Gradient Program:

| Time (min) | % A | % B |
|---|---|---|
| 0:00 | 95 | 5 |
| 8:00 | 65 | 35 |
| 12:00 | 35 | 65 |
| 15:00 | 0 | 100 |
| 16:00 | 95 | 5 |
| 18:00 | 95 | 5 |

Detection can be carried out conveniently by ultraviolet (UV) wavelength@230 nm. Quantitation Limit is the lowest amount of (S)-7,8-saturated-4,5-epoxy-morphinaniums that can be consistently measured and reported, regardless of variations in laboratories, analysts, instruments or reagent lots. Detection Limit is the lowest amount of (S)-7,8-saturated-4,5-epoxy-morphinaniums in a sample which can be detected but not necessarily quantitated as an exact value.

The foregoing HPLC also can be used to determine the relative amount of (R)-7,8-saturated-4,5-epoxy-morphinanium and its (S) stereoisomer and the intermediates of the synthesis thereof by determining the area under the respective (R) and (S) curves in the chromatogram produced. According to another aspect of the invention a method for isolation and purification of as (R)-7,8-saturated-4,5-epoxy-morphinanium and the 3-0-protected-(R)-7,8-saturated-4,5-epoxy-morphinanium salt intermediate is provided, comprising recrystallizing the crude (R)-7,8-saturated-4,5-epoxy-morphinanium or intermediates thereof from a solvent or a mixture of solvents. This process can be in addition to the method described above, after the deprotection step and/or the anion exchange resin column step.

The pharmaceutical preparations of the invention can take on a variety of forms, including, but not limited to a composition that is enteric coated, a composition that is a controlled release or sustained release formulation, a composition that is a solution, a composition that is a topical formulation, a composition that is a suppository, a composition that is lyophilized, a composition that is in an inhaler, a composition that is in a nasal spray device, and the like. The composition can be for oral administration, parenteral administration, mucosal administration, nasal administration, topical administration, ocular administration, local administration, etc. If parenteral, the administration can be subcutaneous, intravenous, intradermal, intraperitoneal, intrathecal, etc. The pharmaceutical preparation may be in a packaged unit dosage or multi-unit dosage.

According to one aspect of the invention a pharmaceutical composition is provided that comprises a (R)-7,8-saturated-4,5-epoxy-morphinanium free of its (S)-7,8-saturated-4,5-epoxy-morphinanium counterpart, as detectable by the chromatography procedures described herein, or comprises the 3-0-protected-(R)-7,8-saturated-4,5-epoxy-morphinanium salt intermediate free of this (S) counterpart, and a pharmaceutically acceptable carrier.

Certain embodiments entail purification of the salt of the (R)-7,8-saturated-4,5-epoxy-morphinanium by chromatography, recrystallization, or a combination thereof. In one embodiment, the purification is by multiple recrystallizations.

According to yet another aspect of the invention, a pharmaceutical preparation containing an (R)-7,8-saturated-4,5-epoxy-morphinanium, or the 3-0-protected-(R)-7,8-saturated-4,5-epoxy-morphinanium salt intermediate, in a lyophilized formulation is prepared by combining a cryoprotective agent, such as mannitol, with the (R)-7,8-saturated-4,5-epoxy-morphinanium formulation. The lyophilized preparation may also contain any one of, any combination of, or all of a buffering agent, an antioxidant, and an isotonicity agent and an opioid. In one embodiment the aforementioned pharmaceutical composition can further comprise one pharmaceutical agent that is not an opioid antagonist. In one embodiment of the invention the aforementioned pharmaceutical composition can comprise a pharmaceutical agent. In yet another embodiment, the pharmaceutical composition can further comprise at least one opioid, and at least one pharmaceutical agent that is not an opioid or an opioid antagonist. In an embodiment the pharmaceutical agent that is not an opioid or an opioid antagonist is a non-opioid analgesic/anti-pyretic such as acetaminophen, an antiviral agent, an anti-infective agent, an anticancer agent, an antispasmodic agent, an anti-muscarinic agent, a steriodal or non-steriodal anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, a herbal medicine, an anti-diarrheal medication, a laxative, a stool softener, a fiber or a hematopoietic stimulating agent.

The pharmaceutical compositions of the invention can be provided in kits. The kits are a package containing a sealed container comprising the pharmaceutical preparations of the present invention and instructions for use. The kits contain a (R)-7,8-saturated-4,5-epoxy-morphinanium that is free of HPLC detectable (S) counterpart stereoisomer. The kit in one embodiment contains 40 mg/mL the (R)-7,8-saturated-4,5-epoxy-morphinanium compound. The kit in another embodiment contains 30 mg/mL of (R)-7,8-saturated-4,5-epoxy-morphinanium compound. The kit may further include an opioid or opioid agonist, or it can include at least one pharmaceutical agent that is not an opioid or an opioid antagonist. In one embodiment, the kit is a package containing a sealed container comprising the pharmaceutical preparation that is or the 3-0-protected-(R)-7,8-saturated-4,5-epoxy-morphinanium salt and instructions for use. The kit in one embodiment contains 40 mg/mL 3-0-protected-(R)-7,8-saturated-4,5-epoxy-morphinanium salt. The kit in another embodiment contains 30 mg/mT of 3-0-protected-(R)-7,8-saturated-4,5-epoxy-morphinanium salt. The kit can further include an opioid or opioid agonist, or it can include at least one pharmaceutical agent that is not an opioid or an opioid antagonist.

According to another aspect of the invention, methods are provided for ensuring the manufacture of (R)-7,8-saturated-4,5-epoxy-morphinaniums of the present disclosure (which are opioid antagonists) that is free of their (S)-7,8-saturated-4,5-epoxy-morphinanium stereoisomers (which are opioid agonists). The methods permit for the first time the assurance that a pharmaceutical preparation of a (R)-7,8-saturated-4,5-epoxy-morphinanium which is intended for antagonist activity is not contaminated with a compound that opposes the activity of (R)-7,8-saturated-4,5-epoxy-morphinanium. This is particularly desirable when the (R)-7,8-saturated-4,5-epoxy-morphinanium is administered to oppose the side effects of opioid therapy, as opioids generally appear to act synergistically with (S)-7,8-saturated-4,5-epoxy-morphinaniums to oppose the activity of (R)-7,8-saturated-4,5-epoxy-morphinanium.

In an embodiment, a method is provided for manufacturing an (R)-7,8-saturated-4,5-epoxy-morphinanium. The method entails: (a) obtaining a first composition containing an (R)-7,8-saturated-4,5-epoxy-morphinaniums, (b) purifying the first composition by chromatography, recrystallization or a combination thereof, (c) conducting HPLC on a sample of purified first composition using the (S)-7,8-saturated-4,5-epoxy-morphinanium counterpart stereoisomer as a standard, and (d) determining the presence or absence of the (S)-7,8-saturated-4,5-epoxy-morphinanium in the sample. In one embodiment, both the (R)-7,8-saturated-4,5-epoxy-morphinanium and its counterpart (S)-7,8-saturated-4,5-epoxy-morphinanium stereoisomer are used as standards, to determine for example relative retention time of the (R)-7,8-saturated-4,5-epoxy-morphinanium and (S)-7,8-saturated-4,5-epoxy-morphinanium. In one embodiment, the purifying is multiple recryallization steps or multiple chromatography steps. In another embodiment, the purifying is carried out until (S)-7,8-saturated-4,5-epoxy-morphinanium is absent from the sample as determined by HPLC. It should be understood, however, that the purified first composition in some aspects of the invention is not necessarily free of detectable (S)-7,8-saturated-4,5-epoxy-morphinanium. The presence of such (S)-7,8-saturated-4,5-epoxy-morphinanium, for example, might indicate that further purification steps should be conducted if a purer (R)-7,8-saturated-4,5-epoxy-morphinanium is desired.

The methods can further involve packaging purified first composition that is free of HPLC a detectable (S)-7,8-saturated-4,5-epoxy-morphinanium. The methods further can include providing indicia on or within the packaged, purified first composition indicating that the packaged, purified first composition is free of the HPLC detectable (S)-7,8-saturated-4,5-epoxy-morphinanium. The method further can involve packaging a pharmaceutically effective amount for treating anyone of the conditions described herein. The first composition containing an (R)- and (S)-7,8-saturated-4,5-epoxy-morphinanium can be obtained by the methods described herein.

According to one aspect of the invention, the purifying is carried out until (S)-7,8-saturated-4,5-epoxy-morphinanium is less than 0.4%, 0.3%, 0.2%, 0.15%, 0.1%, 0.05%, even is absent from the purified first composition as determined by HPLC with a detection limit of 0.02 and a quantization limit of 0.05%. In one embodiment the method provides indicia on or with the packaged purified first composition indicating a level of (S)-7,8-saturated-4,5-epoxy-morphinaniums in the packaged first purified composition.

According to one aspect of the invention a package is provided that contains a composition comprising a (R)-7,8-saturated-4,5-epoxy-morphinanium and indicia on or contained within the package indicating a level of counterpart (S)-7,8-saturated-4,5-epoxy-morphinanium in the composition. In one embodiment the level of (S)-7,8-saturated-4,5-epoxy-morphinanium is less than 0.4%, 0.3%, 0.2%, 0.15%, 0.1%, 0.05%, or is absent from the sample. In yet another embodiment, the package further contains, mixed together with the (R)-7,8-saturated-4,5-epoxy-morphinanium, one or more of a buffering agent, a chelating agent, a preserving agent, a cryoprotecting agent, a lubricating agent, a preservative, an anti-oxidant, or a binding agent.

According to one aspect of the invention a method of preparing a pharmaceutical product in provided, by selecting a composition of (R)-7,8-saturated-4,5-epoxy-morphinanium because it contains (S)-7,8-saturated-4,5-epoxy-morphinanium at a level that is less than 0.4%, 0.3%, 0.2%, 0.15%, 0.1%, 0.05% of, or is absent from the composition, and formulating the composition into a unit or multi unit dosage for administration to a patient.

According to another aspect of the invention, a packaged product is provided. The package contains a composition comprising a (R)-7,8-saturated-4,5-epoxy-morphinanium, wherein the composition is free of HPLC detectable (S)-7,8-saturated-4,5-epoxy-morphinanium counterpart stereoisomer, and indicia on or contained within the package indicating that the composition is free of the HPLC detectable (S)-7,8-saturated-4,5-epoxy-morphinanium. The composition can take on a variety of forms, including, but not limited to, a standard for use in laboratory experiments, a standard for use in manufacturing protocols, or a pharmaceutical composition. If the composition is a pharmaceutical composition, then one form of indicia is writing on a label or package insert describing the characteristics of the pharmaceutical preparation. The indicia can indicate directly that the composition is free of a (S)-7,8-saturated-4,5-epoxy-morphinanium, or it can indicate the same indirectly, by stating for example that the composition is pure or 100% a particular (R)-7,8-saturated-4,5-epoxy-morphinanium. The pharmaceutical composition can be for treating any of the conditions described herein. The pharmaceutical composition can contain an effective amount of the pure (R)-7,8-saturated-4,5-epoxy-morphinanium and can take any of the forms described below as if specifically recited in this summary, including, but not limited to, solutions, solids, semi-solids, enteric coated materials and the like.

According to embodiment, a method is provided for treating or preventing opioid-induced side effects comprising administering to a patient a physiological concentration of (R)-7,8-saturated-4,5-epoxy-morphinanium of the present invention free of detectable (S)-stereoisomer by the chromatography procedures described herein, or the 3-0-protected-(R)-7,8-saturated-4,5-epoxy-morphinanium salt intermediate composition of any of the foregoing aspects of the invention, in an amount effective to treat the opioid-induced side effect. At physiological concentrations, (R)-7,8-saturated-4,5-epoxy-morphinaniums of the present disclosure have been found to have opioid antagonist activity with little, if any, agonist activity.

In one embodiment of the invention, the patient is chronically administered opioids. In another embodiment the patient is acutely administered opioids. The opioid-induced side effect is preferably selected from a group consisting of constipation, immune suppression, inhibition of gastrointestinal motility, inhibition of gastric emptying, nausea, emesis, incomplete evacuation, bloating, abdominal distension, increased gastroesophageal reflux, hypotension, bradycardia, gastrointestinal dysfunction, pruritus, dysphoria, and urinary retention. In one embodiment the opioid-induced side effect is constipation. In another embodiment the opioid-induced side effect is inhibition of gastrointestinal motility or inhibition of gastric emptying. In yet another embodiment the opioid-induced side effect is nausea or emesis. In yet another embodiment the opioid-induced side effect is pruritus. In yet another embodiment the opioid-induced side effect is dysphoria. In yet another embodiment the opioid-induced side effect is urinary retention.

According to embodiment, a method is provided for treating a patient receiving an opioid for pain resulting from surgery comprising administering to the patient an (R)-7,8-saturated-4,5-epoxy-morphinanium (or the 3-0-protected-(R)-7,8-saturated-4,5-epoxy-morphinaniums salt intermediate) composition free of it detectable (S)-7,8-saturated-4,5-epoxy-morphinanium stereoisomer by the chromatography procedures described herein in an amount effective to promote gastrointestinal motility, gastric emptying or relief of constipation.

According to another aspect of the invention, a method is provided for inducing Taxation in a patient in need of Taxation, comprising administering to the patient an (R)-7,8-saturated-4,5-epoxy-morphinanium or the 3-0-protected intermediate-(R)-7,8-saturated-4,5-epoxy-morphinanium salt composition free of detectable (S)-counterpart stereoisomer by the chromatography procedures described herein in an effective amount.

According to yet another aspect of the invention, a method is provided for preventing and/or treating impaction in a patient in need of such prevention/treatment, comprising administering to the patient an (R)-7,8-saturated-4,5-epoxy-morphinanium (or the 3-0-protected-(R)-7,8-saturated-4,5-epoxy-morphinanium salt intermediate) composition of the present disclosure free of detectable counterpart (S)-7,8-saturated-4,5-epoxy-morphinanium by the chromatography procedures described herein or in an effective amount.

According to yet another aspect of the invention, a method is provided for preventing and/or treating post-operative bowel dysfunction following surgery, in particular abdominal surgery in a patient in need of such prevention/treatment, comprising administering to the patient an (R)-7,8-saturated-4,5-epoxy-morphinanium composition (or the 3-0-protected-(R)-7,8-saturated-4,5-epoxy-morphinaniums salt intermediate) of the present disclosure free of it (S)-7,8-saturated-4,5-epoxy-morphinanium stereoisomeric counterpart as detectable by the chromatography procedures described herein in an effective amount.

According to one aspect of the invention, a method is provided for treating or preventing endogenous opioid-induced dysfunction comprising administering to the patient an (R)-7,8-saturated-4,5-epoxy-morphinanium of the disclosure, or the 3-0-protected-(R)-7,8-saturated-4,5-epoxy-morphinaniums salt intermediate thereof, free of its (S)-7,8-saturated-4,5-epoxy-morphinanium stereoisomer, as judged by detection by the chromatography procedures described herein, in an amount effective to treat the endogenous opioid-induced gastrointestinal dysfunction. The dysfunction can be selected from the group consisting of gastrointestinal dysfunction, obesity, hypertension, and addiction. The gastrointestinal dysfunction can be selected from a group consisting of inhibition of gastrointestinal motility, constipation and ileus. In some embodiments of the invention the ileus is selected from the group comprising of: post-operative ileus, post-partum ileus, paralytic ileus.

According to one aspect of the invention, a method is provided for preventing or treating idiopathic constipation comprising administering to the patient an (R)-7,8-saturated-4,5-epoxy-morphinaniums composition free of detectable (S)-7,8-saturated-4,5-epoxy-morphinaniums by the chromatography procedures described herein the 3-0-protected-(R)-7,8-saturated-4,5-epoxy-morphinaniums salt intermediate in an amount effective to prevent or treat the idiopathic constipation.

According to yet another aspect of the invention, a method is provided for treating irritable bowel syndrome comprising administering to the patient an (R)-7,8-saturated-4,5-epoxy-morphinanium composition (or the 3-0-protected-(R)-7,8-saturated-4,5-epoxy-morphinanium salt intermediate thereof) free of detectable (S)-7,8-saturated-4,5-epoxy-morphinanium by the chromatography procedures described herein in an amount effective to ameliorate at least one symptom of the irritable bowel syndrome. In some embodiments of the invention the (R)-7,8-saturated-4,5-epoxy-morphinanium composition, or the 3-0-protected-(R)-7,8-saturated-4,5-epoxy-morphinaniums salt composition, further comprises at least one irritable bowel syndrome therapeutic agent. The irritable bowel syndrome therapeutic agent can be selected from the groups consisting of antispasmodics, anti-muscarinics, anti-inflammatory agents, pro-motility agents, 5HT₁ agonists, 5HT₃ antagonists, 5HT₄ antagonists, 5HT₄ agonists, bile salt sequestering agents, bulk-forming agents, alpha2-adrenergic agonists, mineral oils, antidepressants, herbal medicines, anti-diarrheal medication and combinations thereof.

Compounds of the present invention may also find use in attenuating endothelial cell proliferation, preventing unwanted angiogensis (particularly in cancer compromised individuals, and in diabetes, sickle cell anemia, vascular wound, unwanted ocular neovascularization, proliferative retinopathy), inhibition of VEGF activity in endothelial cells, inhibiting Rho A and activation in endothelial cells, when administered alone and/or in combination with other drugs (including, without limitation, methylnaltrexone and other opioid compounds). Such compounds further may be used to reduce opioid side-effects as set forth above, including (without limitation) dysphoria, pruritis, urinary retention, nausea, emesis, opioid-induced immune suppression.

According to one aspect of the invention. a method is provided for treating obesity comprising administering to the patient an axial N-oxide-4,5-epoxy-morphinanium composition (or the 3-0-protected equatorial N-oxide-4,5-epoxy-morphinanium salt intermediate thereof) free of detectable equatorial N-oxide stereoisomer by the chromatography procedures described herein in an amount effective to ameliorate obesity. In some embodiments of the invention the axial N-oxide-4,5-epoxy-morphinanium composition, or the 3-0-protected axial N-oxide-4,5-epoxy-morphinanium composition, further comprises at least one weight-management drug, such as anti-obesity drugs. An anti-obesity drug includes, without limitation, orlistat, sibutramine, metformin, byetta, symlin, rimonabant, pyruvate, and phenylpropanolamine.

According to one aspect of the invention methods are provided for parenteral administration of the compounds and compositions of the invention including but not limited to intravenous, intramuscular and subcutaneous administration. In one embodiment of the invention the compounds of the invention are in pharmaceutical preparations suitable for use in pre-filled syringes, pre-filled pen injectors, cartridges for use in pen injectors, reusable syringes or other medical injectors, liquid dry injectors, needleless pen systems, syrettes, autoinjectors, or other patient-controlled injection devices.

These and other aspects of the invention are described in greater detail herein.

### BRIEF DESCRIPTIONS OF DRAWINGS

FIG. 1 provides one of the potential structures of a 7,8-saturated-4,5-epoxy-morphinanium embodiment of the present invention.

FIG. 2 provides a proton NMR spectrum of (S)-17-allyl-17-cyclopropylmethyl-4,5α-epoxy-3,14-dihydroxy-6-oxomorphinanium iodide.

FIG. 3 provides a proton NMR spectrum of (R)-17-allyl-17-cyclopropylmethyl-4,5α-epoxy-3,14-dihydroxy-6-oxomorphinanium iodide.

FIG. 4 provides a proton NMR spectrum of (R)-17-cyclobutylmethyl-4,5α-epoxy-3,14-dihydroxy-17-methyl-6-oxomorphinanium iodide.

FIG. 5 provides a proton NMR spectrum of (R)-17-cyclopropylmethyl-4,5α-epoxy-3,14-dihydroxy-17-methyl-6-methylenemorphinanium iodide.

### DETAILED DESCRIPTION OF THE INVENTION

The invention provides for (R)-7,8-saturated-4,5-epoxy-morphinanium compounds, synthetic routes for stereoselective synthesis of (R)-7,8-saturated-4,5-epoxy-morphinanium compounds, substantially pure (R)-7,8-saturated-4,5-epoxy-morphinanium compounds, crystals of substantially pure (R)-7,8-saturated-4,5-epoxy-morphinanium compounds, methods of analysis of (R)-7,8-saturated-4,5-epoxy-morphinanium compounds, pharmaceutical preparations containing substantially pure (R)-7,8-saturated-4,5-epoxy-morphinanium compounds, and methods for their use.

(R)-7,8-saturated-4,5-epoxy-morphinaniums of the present invention include the structure of Formula Z:

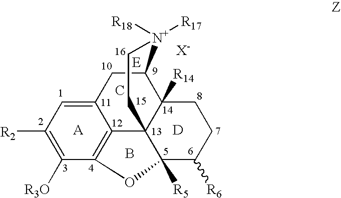

wherein X is a counterion and R₁₇ and R₁₈ are selected to result in an (R) configuration about the nitrogen in conformity with the Cahn, Ingold, Prelog configuration assignment rules, and R₁₈ and R₁₇ are C₁-C₈ alkyls or C₁-C₆ alkyls. R₃ may be a hydroxyl protecting group. The counterion can be any counterion, including a zwitterion. Preferably the counterion is pharmaceutically acceptable. Counterions include halides, sulfates, phosphates, nitrates, and anionic-charged organic species. The halide can be iodide, bromide, chloride, fluoride, or combinations thereof. In one embodiment the halide is iodide. In one embodiment the halide is bromide. The anionic-charged organic species may be a sulfonate or carboxylate.

Included are the (R)—N-configuration compounds of the formula I:

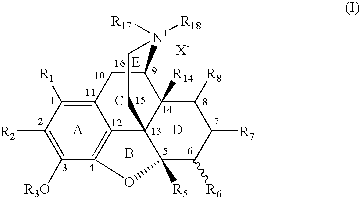

or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof; or R₁ and R₂ can also be combined to form a C₃-C₆ carbocycle fused ring which may be substituted according to R₁₉, a benzo fused ring, or a 5-6 membered heteroaryl fused ring;
R₃ is H, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀; (C₃-C₁₀) carbocycle substituted with 0-3R₂₀;
   aryl substituted with 0-3R₂₀;
   C₁-C₃ acyl
R₅ is H, OH, OR₂₆,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, OH, OR₂₆;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   amine, amide, sulfonamide, or ester;
R₇ and R₈ are independently H, hydrocarbyl, cyclohydrocarbyl, or substituted moieties thereof; or R₇ and R₈ are combined to form a carbocycle fused ring which may be substituted according to R₁₉, a benzo fused ring, or a 5-6 membered heteroaryl fused ring;
R₁₄ is H, OH, OR₂₆, NR₂₂R₂₃SR₂₅, S(═O)R₂₅, SO₂R₂₅;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀; aryloxy, acyloxy,
   or R₁₄ can be combined with R₁₇ or R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl;
R₁₉ is at each occurrence is independently selected from:
   H, C₁-C₆ alkyl, CF₃, OR₂₄, Cl, F, Br, I, ═O, CN, NO₂, NR₂₂R₂₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂,
   NR₂₂R₂₃, acetyl,
   C₁-C₆ alkyl, C₁-C₄ alkoxy, C₁-C₄ haloalkyl, C₁-C₄ haloalkoxy, and C₁-C₄ haloalkyl-S—;
R₂₁, at each occurrence, is independently selected from H, OH, Cl, F, Br, I, CN, NO₂,
   NR₂₂R₂₃, CF₃, acetyl,
   C₁-C₆ alkyl, C₁-C₄ alkoxy, C₁-C₄ haloalkyl, C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, (C₁-C₆ alkyl)-C(═O)—, and (C₁-C₆ alkyl)-S(═O)₂—;
R₂₃, at each occurrence, is independently selected from:
   H, (C₁-C₆)alkyl,
   (C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from
   H, C₁-C₆ alkyl, CF₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion

Certain groups may be preferentially chosen. For example, R₁₄ may be selected to be OH or O-alkyl in one embodiment.

Included in embodiments herein are the isolated (R)-stereoisomers of the formula Ia:

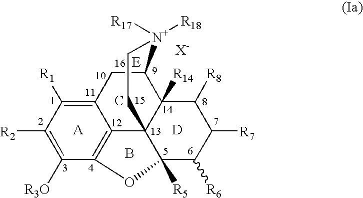

wherein
R₁₇ and R₁₈ are selected alternatively with respect to one another from (a) or (b):
   (a) unsubstituted or non-halogen substituted: C₄-C₈ (cycloalkyl)alkyl or (cycloalkenyl)alkyl, (cycloheteryl)alkyl, (cycloaryl)alkyl; C₄-C₆ (cycloalkyl)alkyl or (cycloalkenyl)alkyl, (cycloheteryl)alkyl, (cycloaryl)alkyl
   (b) substituted or unsubstituted linear or branched C₁-C₃ alkyl, C₂-C₃ alkenyl, or C₃-alkynyl;
   wherein if (b) is selected as methyl, and R₆ is ═O, (a) is not unsubstituted (cyclopropyl)methyl;
R₆ is H, OH, ═O, ═CH₂, —N(CH₃)₂, or any cyclic ring, or forms a cyclic ring with R₇;
R₇ and R₈ are H or alkyl;
R₁₄ is H, OH, halide, arylamido, amino, N-alkyl, N-dialkyl, N-aryl, N-alkylaryl, N-cycloalkylalkyl, SCH₃, S(═O)CH₃, S(═O)₂CH₃, alkoxy, aryloxy, or aryl-alkoxy or forms a cyclic ring with R₁₇ or R₁₈;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

Included in embodiments herein are the (R)-stereoisomers of the formula Ib:

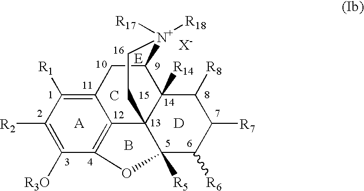

wherein
R₁₇ and R₁₈ are a substituted or unsubstituted C₁-C₆ hydrocarbyl, wherein when R₆ is selected as ═O, at least one of which is not methyl when the other is cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₈ are H or hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, SR₂₅, S(═O)R₂₅, SO₂R₂₅; alkoxy, aryloxy, or arylalkoxy, or forms a ring with R₁₇ or R₁₈;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.

An isolated compound of the (R) configuration with respect to the nitrogen of Formula I(c):

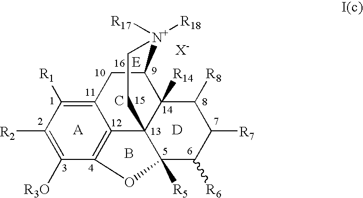

or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof;
   or R₁ and R₂ can also be combined to form a C₃-C₆ carbocycle fused ring which may be substituted according to R₁₉, a benzo fused ring, or a 5-6 membered heteroaryl fused ring;
R₃ is H, silyl, CO₂R₁₉, SO₂R₁₉, B(OR₂₆)₂;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   C₁-C₃ acyl
R₅ is H, OH, OR₂₆,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, OH, OR₂₆, =(R₁₉)(R_{19′}), =(hetero cycle substituted with 0-3R₂₀), =(C₃-C₇ cycle substituted with 0-3R₂₀);
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   amine, amide, sulfonamide, or ester;
R₇ and R₈ are independently H, hydrocarbyl, cyclohydrocarbyl, hetero cycle with 0-3R₂₀, alkylaryl with 0-3R₂₀, arylakly with 0-3 R₂₀, or substituted moieties thereof, or

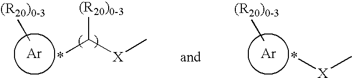

where, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
or R₇ and R₈ are combined to form a carbocycle fused ring which may be substituted according to R₁₉, a benzo fused ring, 5-, 6-, or a 5-6 membered aryl or heteroaryl with 0-3R₂₀;
R₁₄ is H, OH, OR₂₆, NR₂₂R₂₃SR₂₅, S(═O)R₂₅, SO₂R₂₅, hetero cycle with 0-3R₂₀, alkylaryl with 0-3R₂₀, arylalkyl with 0-3R₂₀,

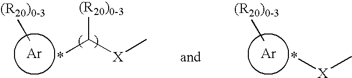

wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl, hetero cycle with 0-3R₂₀, alkylaryl with 0-3R₂₀, arylalkyl with 0-3_{R20},

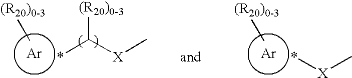

wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
R₁₉ is at each occurrence is independently selected from: H, C₁-C₆ alkyl, CF₃, OR₂₄, Cl, F, Br, I, ═O, CN, NO₂, NR₂₂R₂₃ aryl substituted with 0-3R₂₀;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂,
   NR₂₂R₂₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂,
   NR₂₂R₂₃, CF₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, hetero aryl, hetero cycle, alkylaryl, and arylalkyl;
   (C₁-C₆ alkyl)-C(═O)—, and (C₁-C₆ alkyl)-S(═O)₂—;
R₂₃, at each occurrence, is independently selected from: H, (C₁-C₆)alkyl, C₆-C₁₀ aryl, hetero aryl, hetero cycle, alkylaryl, haloalkyl, arylalkyl,
   (C₁-C₆ alkyl)—C(═O)—, and (C₁-C₆ alkyl)-S(—O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from:
   H, C₁-C₆ alkyl, CF₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

Also included in the present invention are the (R)-isomers of the compounds disclosed in U.S. Pat. No. 6,713,488 to Sadee et al. and U.S. Patent Publication No. 2006/0014771, which are incorporated by reference where appropriate for teachings of additional or alternative details, features and/or technical background. Applicants' discern no teaching in either reference of the different pharmacological actions seen with respect to the (R) and (S) stereoisomers of the compounds disclosed in these references.

The invention is intended to embrace (R)-quaternary derivatives of noroxymorphone where the cyclopropylmethyl is replaced with a moiety (Q), where (Q) is a 1-20 carbon hydrocarbyl consisting exclusively of carbon and hydrogen, including alkyl, alkenyl, alkynyl, and aryl, substituted or unsubstituted with hydrocarbons or with one or more atoms such as nitrogen, oxygen, silicon, phosphorus, boron, sulfur, or halogen (described in PCT publication WO 2004/043964.) In embodiments, (Q) is allyl, chloroallyl, or propargyl. In other embodiments, the hydrocarbyl contains 4-10 carbons.

The term “acyl”, whether used alone, or within a term such as “acylamino”, denotes a radical provided by the residue after removal of hydroxyl from an organic acid. The term “acylamino” embraces an amine radical substituted with an acyl group. An examples of an “acylamino” radical is acetylamine (CH₃C(═O)—NH—). The term “aryloxy” denotes a radical provided by the residue after removal of hydrido from a hydroxy-substituted aryl moiety (e.g., phenol).

As used herein, “alkanoyl” refers to a-C (═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

The term “alkenyl” includes unsaturated aliphatic groups analogous in length and possible substitution to the alkyls described above, but that contain at least one double bond and must contain at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “lower alkylene” herein refers to those alkylene groups having from about 1 to about 6 carbon atoms. The term “alkenyl” includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkenylene”, in general, refers to an alkylene group containing at least one carbon-carbon double bond. Exemplary alkenylene groups include, for example, ethenylene (—CH═CH—) and propenylene (—CH═CHCH₂—). Alkenylene groups have from 2 to about 4 carbons.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro chloro or bromo to provide “haloalkoxy” or “haloalkoxyalkyl” radicals. Examples of “alkoxy” radicals include methoxy butoxy and trifluoromethoxy.

“Alkyl” in general, refers to an aliphatic hydrocarbon group which may be straight, branched or cyclic having from 1 to about 10 carbon atoms in the chain, and all combinations and subcombinations of ranges therein, e.g., a cycloalkyl, branched cycloalkylalkyl, a branched alkylcycloalkyl having 4-10 carbon atoms. The term “alkyl” includes both “unsubstituted alkyls” and “substituted alkyls,” the latter of which refers to alkyl moieties having substituents replacing a hydrogen on one or more carbons of the backbone. “Lower alkyl” refers to an alkyl group having 1 to about 6 carbon atoms. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2-dimethylbutyl, and 2,3-dimethylbutyl, cyclopropylmethyl and cyclobutylmethyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. The term “aralkyl” embraces aryl-substituted alkyl radicals such as benzyl, diphenylmethyl, triphenylmethyl, phenethyl, phenylpropyl, and diphenethyl. The terms benzyl and phenylmethyl are interchangeable. The term “n-alkyl” means a straight chain (i.e. unbranched) unsubstituted alkyl group. “Branched” refers to an alkyl group in which a lower alkyl group, such as methyl, ethyl or propyl, is attached to a linear alkyl chain.

An “alkylating agent” is a compound that can be reacted with a starting material to bind, typically covalently, an alkyl group to the starting material. The alkylating agent typically includes a leaving group that is separated from the alkyl group at the time of attachment to the starting material. Leaving groups may be, for example, halogens, halogenated sulfonates or halogenated acetates. An example of an alkylating agent is cyclopropylmethyl iodide.

The term “alkylsilyl” denotes a silyl radical substituted with an alkyl group. The term “alkylsilyloxy” denotes a silyloxy radical (—O—Si—) substituted with an alkyl group. An example of an “alkylsilyloxy” radical is —O—Si-t-BuMe₂.

The term “alkylsulfinyl” embraces radicals containing a linear or branched alkyl radical, of one to ten carbon atoms, attached to a divalent—S(═O)— atom. The term “arylsulfinyl” embraces aryl radicals attached to a divalent—S(═O)— atom (e.g., —S═OAr).

The term “alkylthio” embraces radicals containing a linear or branched alkyl radical, of one to ten carbon atoms, attached to a divalent sulfur atom. The term “arylsulfenyl” embraces aryl radicals attached to a divalent sulfur atom (—SAr) An example of “alkylthio” is methylthio, (CH₃—(S)—).

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups.

The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkyl radicals substituted with an N-alkyl-N-hydroxyamido radical. The term “amidoalkyl” embraces alkyl radicals substituted with amido radicals.

The term “aminoalkyl” embraces alkyl radicals substituted with amine radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl.

“Aryl-substituted alkyl”, in general, refers to an linear alkyl group, preferably a lower alkyl group, substituted at a carbon with an optionally substituted aryl group, preferably an optionally substituted phenyl ring. Exemplary aryl-substituted alkyl groups include, for example, phenylmethyl, phenylethyl and 3-(4-methylphenyl)propyl.

The term “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred “carbocycle” are cyclopropyl, cyclobutyl, cyclopentyl, and cyclohexyl.

The term “cycloalkyl” embraces radicals having three to ten carbon atoms, such as cyclopropyl cyclobutyl, cyclopentyl, cyclohexyl and cycloheptyl.

“Cycloalkyl-substituted alkyl”, in general, refers to a linear alkyl group, preferably a lower alkyl group, substituted at a terminal carbon with a cycloalkyl group, preferably a C₃-C₈ cycloalkyl group. Typical cycloalkyl-substituted alkyl groups include cyclohexylmethyl, cyclohexylethyl, cyclopentylethyl, cyclopentylpropyl, cyclopropylmethyl and the like.

“Cycloalkenyl”, in general, refers to an olefinically unsaturated cycloalkyl group having from about 4 to about 10 carbons, and all combinations and subcombinations of ranges therein. In some embodiments, the cycloalkenyl group is a C₅-C₈ cycloalkenyl group, i.e., a cycloalkenyl group having from about 5 to about 8 carbons.

“Dipolar aprotic” solvents are protophilic solvents that cannot donate labile hydrogen atoms and that exhibit a permanent dipole moment. Examples include acetone, ethyl acetate, dimethyl sulfoxide (DMSO), dimethyl formamide (DMF) and N-methylpyrrolidone.

“Dipolar protic” solvents are those that can donate labile hydrogen atoms and that exhibit a permanent dipole moment. Examples include water, alcohols such as 2-propanol, ethanol, methanol, carboxylic acids such as formic acid, acetic acid, and propionic acid.

The phrase “does not substantially cross,” as used herein, means that less than about 20% by weight of the compound employed in the present methods crosses the bloodbrain barrier, preferably less than about 15% by weight, more preferably less than about 10% by weight, even more preferably less than about 5% by weight and most preferably 0% by weight of the compound crosses the blood-brain barrier.

The term “halo” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals.

As used herein, the term “heterocycle” or “heterocyclic ring” is intended to mean a stable 5- to 7-membered monocyclic or bicyclic or 7- to 14-membered bicyclic heterocyclic ring which is saturated, partially unsaturated, or unsaturated (aromatic), and which consists of carbon atoms and 1, 2, 3 or 4 heteroatoms independently selected from the group consisting of N, O and S and including any bicyclic group in which any of the above-defined heterocyclic rings is fused to a benzene ring. Examples of saturated heterocyclic radicals include pyrrolidyl and morpholinyl.

The term “hydroxyalkyl” embraces linear or branched alkyl radicals having one to about ten carbon atoms any one of which may be substituted with one or more hydroxyl radicals.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

The terms “N-alkylamino” and “N,N-dialkylamino” denote amine groups which have been substituted with one alkyl radical and with two alkyl radicals, respectively.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

“Organic solvent” has its common ordinary meaning to those of skill in this art. Exemplary organic solvents useful in the invention include, but are not limited to tetrahydrofuran, acetone, hexane, ether, chloroform, acetic acid, acetonitrile, chloroform, cyclohexane, methanol, and toluene. Anhydrous organic solvents are included.

As used herein, “patient” refers to animals, including mammals, preferably humans.

As used herein, “peripheral” or “peripherally-acting” refers to an agent that acts outside of the central nervous system. As used herein, “centrally-acting” refers to an agent that acts within the central nervous system (CNS). The term “peripheral” designates that the compound acts primarily on physiological systems and components external to the central nervous system. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably 0% of the pharmacological activity of the compounds employed in the present methods is exhibited in the CNS.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, the term “side effect” refers to a consequence other than the one (s) for which an agent or measure is used, as the adverse effects produced by a drug, especially on a tissue or organ system other then the one sought to be benefited by its administration.

As used herein, “stereoisomers” refers to compounds that have identical chemical constitution, but differ as regards the arrangement of the atoms or groups in space.

The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂NH₂). The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical.

“Tertiary amines” has its common, ordinary meaning. In general, the tertiary amines useful in the invention have the general formula:

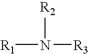

wherein R₁, R₂, and R₃ are identical or a combination of different straight or branched chain alkyl groups, alkenyl groups, alkylene groups, alkenylene groups, cycloalkyl groups, cycloalkyl-substituted alkyl groups, cycloalkenyl groups, alkoxy groups, alkoxy-alkyl groups, acyl groups, aryl groups, aryl-substituted alkyl groups, and heterocyclic groups. Exemplary tertiary amines useful according to the invention are those where R₁₋₃ is an alkyl group of the formula (CₙH₂ₙ₊₁, n=1-4), or aralkyl group of the formula (C₆H₅ (CH₂)ₙ— [n=1-2]. Exemplary tertiary amines useful according to the invention also are cycloalkyl tertiary amines (e.g., N-methylmorpholine, N-methylpyrrolidine, N-methylpiperidine), pyridine and Proton Sponge® (N,N,N′,N′-tetramethyl-1,8-naphthalene).

An (S)-7,8-saturated-4,5-epoxy-morphinanium exhibits properties different from those of its corresponding (R)-7,8-saturated-4,5-epoxy-morphinaniums of the present invention and different properties from a mixture of the (S) and (R) of the particular 7,8-saturated-4,5-epoxy-morphinanium. Those properties may include mobility on chromatography columns, biological and functional activity, and crystal structure. It is believed that the in vivo clearance rate, the side-effect profile, and the like may also differ from one (R)-7,8-saturated-4,5-epoxy-morphinanium of the present invention or mixtures of the (R)-7,8-saturated-4,5-epoxy-morphinanium and the corresponding (S)-7,8-saturated-4,5-epoxy-morphinanium. Pure (S)-7,8-saturated-4,5-epoxy-morphinaniums may behave as agonists of peripheral opioid receptors as, for example, inhibiting gastrointestinal transit. As a consequence, (S)-7,8-saturated-4,5-epoxy-morphinanium activity may be interfered with or antagonized by (R)-7,8-saturated-4,5-epoxy-morphinanium activity in mixtures containing both (R)-7,8-saturated-4,5-epoxy-morphinaniums and (S)-7,8-saturated-4,5-epoxy-morphinaniums. It therefore is highly desirable to have (R)-7,8-saturated-4,5-epoxy-morphinaniums in isolated and substantially pure form.

In one aspect of the invention, methods for the synthesis of (R)-7,8-saturated-4,5-epoxy-morphinanium are provided. An (R)-7,8-saturated-4,5-epoxy-morphinanium may be produced at a purity of greater than or equal to 10%, 20%, 30%, 40%, 50%, 60%, 70%, 75%, 80%, 85%, 90%, 95%, 97%, 98%, 98.5%, 99%, and 99.5% area under the curve (AUC) based on chromatographic techniques. In an embodiment, the purity of an (R)-7,8-saturated-4,5-epoxy-morphinanium is 98% or greater. The amount of a corresponding (S)-7,8-saturated-4,5-epoxy-morphinanium in the purified (R)-7,8-saturated-4,5-epoxy-morphinanium may be less than or equal to about 90%, 80%, 70%, 60%, 50%, 40%, 30%, 20%, 10%, 5%, 3%, 2%, 1%, 0.5%, 0.3%, 0.2%, 0.1% (AUC) or undetectable by chromatographic techniques described herein. It will be appreciated by the skilled artisan that the detection of the methods will depend upon the detection and quantitation limits of the employed technique. Quantitation Limit is the lowest amount of (R)-7,8-saturated-4,5-epoxy-morphinanium that can be consistently measured and reported, regardless of variations in laboratories, analysts, instruments or reagent lots. Detection Limit is the lowest amount of (S)-7,8-saturated-4,5-epoxy-morphinanium in a sample which can be detected but not necessarily quantitated as an exact value. In one embodiment of the invention the detection limit is 0.1% and the quantitation limit is 0.2%. In yet another embodiment the detection limit is 0.02% and the quantitation limit is 0.05%.

Synthesis of a number of 7,8-saturated-4,5-epoxy-morphinaniums of the present invention may be by the direct alkylation of tertiary morphinan, such as oxymorphone. The phenolic OH group of oxymorphone may be unprotected or protected. The (R)-7,8-saturated-4,5-epoxy-morphinanium salt may include a counterion such as iodide, that can then be exchanged for a more preferred counterion, for example, bromide. A useful starting material in the synthesis of number of (R)-7,8-saturated-4,5-epoxy-morphinaniums is disclosed herein as oxymorphone, which may be obtained at about 95% yield through the demethylation of oxycodone, for example, with boron tribromide. Alternatively, the oxymorphone may be obtained through commercial sources.

An alkylation reaction may be performed in a solvent, or solvent system, that may be anhydrous. The solvent system may be a single solvent or may include a combination of two or more solvents. Suitable solvent systems may include dipolar aprotic solvents such as N-methylpyrrolidone (NMP), dimethyl formamide (DMF), hexamethylphosphoramide (HMPA), acetone, 1,4-dioxane and acetonitrile, and dipolar protic solvents such as 2-propanol. Solvent systems may also include dipolar aprotic solvents in combination with aliphatic ethers, such as tetrahydrofuran (THF), 1,2-dimethoxyethane (glyme), diethyleneglycol dimethyl ether (diglyme), 1,4-dioxane, methyl t-butyl ether (methyl 1,1,-dimethylethyl ether, or 2-methyl-2-methoxypropane) diethyl ether, other polar solvents may also be included in some embodiments. For instance, the solvent system may include acetone, methylethylketone, diethylketone (3-pentanone), and t-butylmethylketone(3,3-dimethylbutan-2-one). Alkylation solvent systems may also include aliphatic or alicyclic congeners of any of the compounds disclosed above. Solvent systems may include two or more solvents in any proportion and appropriate proportions for a particular alkylation reaction may be determined through routine experimentation.

The solvent may be used at a ratio of less than, greater than, or equal to about 1, 2, 3, 4, 5, 10 or more volumes. In some cases it may be preferred to minimize the amount of solvent used, such as when product is to be transferred from the solvent using a liquid/liquid extraction or when product is to be crystallized or when the solvent is to be removed from the product.

The alkylating agent may be added to the starting material in various molar ratios, such as less than 8, 12, 16, 20, 24 or greater than 24 equivalents per equivalent of starting material. Reaction efficiency (production of (R)-7,8-saturated-4,5-epoxy-morphinaniums) may be substantially independent of the amount of alkylating agent used in some cases.

In one set of embodiments, alkylation may be performed using the Finkelstein reaction. For example, an alkyl halide, such as cyclopropylmethyl chloride, can be combined with a halide salt, such as sodium iodide, to continuously supply a reactive halogenated alkylating agent, such as cyclopropylmethyl iodide, that is replenished as it is consumed.

Starting materials may be alkylated at atmospheric pressure in an open vessel or under pressure. The reaction may be conducted such that the temperature is maintained or controlled over the reaction time at a prescribed temperature using methods/equipment as are known in the art. One device for maintaining a controlled temperature throughout the alkylation reaction is a heater/chiller unit. Controlling the temperature throughout the alkylation reaction inhibits or reduces temperature fluctuations. The reaction may need to proceed for a number of hours, for example, up to about 22 hours, or 15 to 22 hours, or 16 to 20 hours. Reaction times may in some cases be shortened through the use of microwave irradiation.

In some embodiments, the (R)-7,8-saturated-4,5-epoxy-morphinanium may be isolated from the solvent in which it is produced. For example, the solvent may be removed from a residue containing the (R)-7,8-saturated-4,5-epoxy-morphinanium, or any (R)-7,8-saturated-4,5-epoxy-morphinanium may be transferred from the alkylation solvent to a transfer solvent. Transfer solvents may be polar or non-polar and may have boiling points below 100° C. Transfer solvents may include esters, aldehydes, ethers, alcohols, aliphatic hydrocarbons, aromatic hydrocarbons and halogenated hydrocarbons. Specific transfer solvents include, for example, dioxane, ethyl acetate, isopropyl acetate, methanol, ethanol, dichloromethane, acetonitrile, water, aqueous HBr, heptane, and MTBE.

Any residue obtained from the solvent may be worked up to purify and isolate the (R) product. Purification and isolation may be done using methods known to those skilled in the art, such as by using separation techniques like chromatography, recrystallization, or combinations of various separation techniques as are known the art. In one embodiment, flash chromatography using a C18 column may be used. For example, a CombiFlash™ Sq 16× from ISCO using a Reverse Phase (C18) RediSep column may be used. Analytic HPLC may be performed, for example, on a Phenomenex Prodigy 5 um OD53 100A column and purification performed on a semi-prep Phenomenex Prodigy 5 um OD53 100A column. Different solvents, such as aqueous methanol solvent modified with 0.2% HBr, may be employed with methanol content varying from, for example, about 2.5% to about 50%. The (R)-7,8-saturated-4,5-epoxy-morphinanium may be purified using recrystallization. The process may be repeated until desired purity of product is obtained. In one embodiment, the (R)-7,8-saturated-4,5-epoxy-morphinanium is recrystallized at least two times, three times, or four or more times to achieve the desired level of purity. For example, an (R)-7,8-saturated-4,5-epoxy-morphinanium may be obtained at purities of greater than or equal to 50%, 80%, 85%, 90%, 95%, 97%, 98%, 98.5%, 99.8% (AUC) based on chromatographic techniques. Any impurities may include the starting material, with no detectable (S)-7,8-saturated-4,5-epoxy-morphinanium. Recrystallization may be achieved using a single solvent, or a combination of solvents. In one embodiment, recrystallization is achieved by dissolving (R)-7,8-saturated-4,5-epoxy-morphinanium in a polar solvent, and then adding a less polar cosolvent. In another recrystallization embodiment, (R)-7,8-saturated-4,5-epoxy-morphinanium is purified by recrystallization from a solvent, for example, methanol, and a cosolvent, such as CH₂Cl₂/IPA (6:1). The recrystallization is repeated to achieve desired purity. In one embodiment, the recrystallization solvent may be an organic solvent or a mixture of organic solvents or a mixture of organic solvent(s) plus water. The solvent may be an alcohol, such as a low molecular weight alcohol, e.g., methanol.

The (R)-7,8-saturated-4,5-epoxy-morphinanium, and its derivatives, may be produced in the salt form. Derivatives such as zwitterions of (S)-7,8-saturated-4,5-epoxy-morphinanium are included. The (R)-7,8-saturated-4,5-epoxy-morphinanium may include a positively charged quaternary ammonium group and may be paired with a counterion such as a monovalent or multivalent anion. These anions may include, for example, halides, sulfates, phosphates, nitrates and charged organic species such as sulfonates and carboxylates. Preferred anions include halides such as bromide, chloride, iodide, fluoride, and combinations thereof. In some embodiments, bromide is most preferred. Specific anions may be chosen based on factors such as, for example, reactivity, solubility, stability, activity, cost, availability and toxicity.

Counterions of the (R)-7,8-saturated-4,5-epoxy-morphinanium salt can be exchanged for alternative counterions. When an alternative counterion is desired, an aqueous solution of an (R)-7,8-saturated-4,5-epoxy-morphinanium salt can be passed over an anion exchange resin column to exchange some or all of the counterion of the (R)-7,8-saturated-4,5-epoxy-morphinanium salt for a preferred alternative counterion. Examples of anion exchange resins include AG 1-X8 in a 100 to 200 mesh grade, available from Bio-Rad. In another embodiment, the (S)-7,8-saturated-4,5-epoxy-morphinanium cation can be retained on a cation exchange resin and can then be exchanged by removing the (S)-7,8-saturated-4,5-epoxy-morphinanium from the resin with a salt solution that includes a preferred anion, such as bromide or chloride, forming the desired (S)-7,8-saturated-4,5-epoxy-morphinanium salt in solution.

The (R)-7,8-saturated-4,5-epoxy-morphinaniums of the present invention have numerous utilities. One aspect of the invention is an (R)-7,8-saturated-4,5-epoxy-morphinanium as a chromatographic standard in identifying and distinguishing its counterpart (S)-7,8-saturated-4,5-epoxy-morphinanium from other components in a sample in a chromatographic separation. Another aspect of the invention is the use of an (R)-7,8-saturated-4,5-epoxy-morphinanium as a chromatographic standard in identifying and distinguishing an (R)-7,8-saturated-4,5-epoxy-morphinanium in a mixture containing an (R)-7,8-saturated-4,5-epoxy-morphinanium and an (S)-7,8-saturated-4,5-epoxy-morphinanium counterpart. An isolated (R)-7,8-saturated-4,5-epoxy-morphinanium is also useful in the development of protocols for purifying and distinguishing an (R)-7,8-saturated-4,5-epoxy-morphinanium from an (S)-7,8-saturated-4,5-epoxy-morphinanium in reaction mixtures.

The (R)-7,8-saturated-4,5-epoxy-morphinanium may be provided in a kit form with instruction for its use as a standard. The kit may further comprise an authentic (S)-7,8-saturated-4,5-epoxy-morphinanium as a standard. The (R)-7,8-saturated-4,5-epoxy-morphinanium for use as a standard preferably has a purity of 99.8% or greater with no detectable stereoisomeric (S)-7,8-saturated-4,5-epoxy-morphinanium.

One embodiment of the invention is a method of resolving and identifying an (R)-7,8-saturated-4,5-epoxy-morphinanium and a counterpart (S)-7,8-saturated-4,5-epoxy-morphinanium in a solution of 7,8-saturated-4,5-epoxy-morphinanium. The (R)-7,8-saturated-4,5-epoxy-morphinanium also is useful in HPLC assay methods of quantifying an amount of an (R)-7,8-saturated-4,5-epoxy-morphinanium in a composition or mixture in which the method comprises applying a sample of the composition or mixture to a chromatography column, resolving the components of the composition or mixture, and calculating the amount of an (R)-7,8-saturated-4,5-epoxy-morphinanium in the sample by comparing the percentage of a resolved component in the sample with the percentage of a standard concentration of an (R)-7,8-saturated-4,5-epoxy-morphinanium. The method is particularly useful in reverse phase HPLC chromatography. The (R)-7,8-saturated-4,5-epoxy-morphinanium of the present invention by virtue of its antagonist activity on opioid receptors, is useful as a standard of agonist activity in in vitro and in vivo opioid receptor assays such as those described herein.

The (R)-7,8-saturated-4,5-epoxy-morphinanium can be used to regulate a condition mediated by one or more peripheral opioid receptors, prophylactically or therapeutically, to antagonize peripheral opioid receptors, in particular peripheral mu opioid receptors. The subjects being administered an (R)-7,8-saturated-4,5-epoxy-morphinanium may receive treatment acutely, chronically or on an as needed basis.

The subjects to which the (R)-7,8-saturated-4,5-epoxy-morphinanium may be administered are vertebrates, in particular mammals. In one embodiment the mammal is a human, nonhuman primate, dog, cat, sheep, goat, horse, cow, pig and rodent. In one embodiment, the mammal is a human.

The pharmaceutical preparations of the invention, when used alone or in cocktails, are administered in therapeutically effective amounts. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, lessen the severity of, or inhibit completely, lessen the progression of, or halt altogether the onset or progression of the condition being treated or a symptom associated therewith. In the case of constipation, an effective amount, for example, is that amount which relieves a symptom of constipation, which induces a bowel movement, which increases the frequency of bowel movements, or which decreases oral-cecal transit time.

The art defines constipation as (i) less than one bowel movement in the previous three days or (ii) less than three bowel movements in the previous week (See e.g., U.S. Pat. No. 6,559,158). In other words, a patient is not constipated (i.e., has “regular bowel movements” as used herein) if the patient has at least one bowel movement every three days and at least three bowel movements per week. Accordingly, at least one bowel movement every two days would be considered regular bowel movements. Likewise, at least one bowel movement per day is a regular bowel movement. Effective amounts therefore can be those amounts necessary to establish or maintain regular bowel movements.

In certain instances, the amount is sufficient to induce a bowel movement within 24 hours of administration of the (R)-7,8-saturated-4,5-epoxy-morphinanium of the present disclosure or the (R)-7,8-saturated-4,5-epoxy-morphinanium intermediate, 3-O-protected-(R)-7,8-saturated-4,5-epoxy-morphinanium salt, 12 hours, 10 hours, 8 hours, 6 hours, 4 hours, 2 hours, 1 hour and even immediately upon administration, depending upon the mode of administration. Intravenous administration may in the appropriate dose produce an immediate effect of Taxation in chronic opioid users. Subcutaneous administration may result in a bowel movement within 12 hours of administration or within 4 hours of administration. When administered to a subject, effective amounts will depend, of course, on the particular condition being treated; the severity of the condition; individual patient parameters including age, physical condition, size and weight; concurrent treatment and, especially, concurrent treatment with opioids where opioids are administered chronically; frequency of treatment; and the mode of administration. These factors are well known to those of ordinary skill in the art and can be addressed with no more than routine experimentation.

Functional constipation is a functional bowel disorder that presents as persistently difficult, infrequent, or seemingly incomplete defecation. Constipating medications, such as opioids and opioid agonists, and in particular extended use of opioids or opioid agonist are contributors to functional constipation. Recently, a Rome III diagnostic criteria was established for functional constipation (Longstreth, G. F. et al, Gastroenterology Vol 130, No. 5, 2006). Under this criteria, the diagnosis of functional constipation is made if the patient has 2 or more of the following symptoms for the last 3 months-with symptom onset at least 6 months prior to diagnosis: a) straining during at least 25% of defecation; b) lumpy or hard stools in at least 25% of defecations, c) sensation of incomplete evacuation for at least 25% of defecations, d) sensation of anorectal obstruction/blockage for at least 25% of defecations, e) manual maneuvers to facilitate at least 25% of defecations (eg., digital evacuation, support of the pelvic floor), f) fewer than 3 defecations per week.

The pharmaceutical preparations of the invention are administered in a therapeutically effective amount to treat or relieve at least one symptom of constipation, for example, the effective amount provides 3 or more defecations per week. In another embodiment, the effective amount treats or relieves two or more symptoms of constipation, for example, the amount is effective to reduce straining during defecation and improve stool consistency; stool consistency rated using the Bristol Stool scores. An improvement in stool consistency indicated by a change from a Type 1 at baseline to a Type 2, preferably a change to a Type 3, Type 4, or Type 5. In an embodiment, the effective amount provides 3 or more defecations per week and improves stool consistency.

Patients amenable to the therapy for opioid agonist induced constipation of the present invention include, but are not limited to, terminally ill patients, patients with advanced medical illness, cancer patients, AIDS patients, post-operative patients, patients with chronic pain, patients with neuropathies, patients with rheumatoid arthritis, patients with osteoarthritis, patients with chronic back pain, patients with spinal cord injury, patients with chronic abdominal pain, patients with chronic pancreatic pain, patients with pelvic/perineal pain, patients with fibromyalgia, patients with chronic fatigue syndrome, patients infected with HCV, patients with irritable bowel syndrome, patients with migraine or tension headaches, patients with sickle cell anemia, patients on hemodialysis, and the like.

Patients amenable to the therapy of the present invention also include, but are not limited to, patients suffering from other dysfunctions caused by opioid agonists, and as well as dysfunctions caused by endogenous opioids, especially in post-operative settings. In certain embodiments, the (R)-7,8-saturated-4,5-epoxy-morphinanium of the present disclosure or intermediate thereof may be employed in an amount sufficient to accelerate discharge from hospital post-surgery, including abdominal surgeries such as rectal resection, colectomy, stomach, esophageal, duodenal, appendectomy, hysterectomy, or non-abdominal surgeries such as orthopedic, trauma injuries, thoracic or transplantation surgery. This treatment may be effective to shorten the length of the time in the hospital, or to shorten the time to a hospital discharge order written post-operatively, for example, by shortening the time to bowel sounds after surgery, or first flatus, to first Taxation or to solid diet intake following surgery compared to an average time to such events in a group of patients who have not been treated with the (R)-7,8-saturated-4,5-epoxy-morphinanium. An (R)-7,8-saturated-4,5-epoxy-morphinanium of the present disclosure, or intermediate thereof, or prodrug thereof, may continue to be provided after the patient has ceased to receive opioid pain medications post-operatively.

Certain patients that may particularly be amenable to treatment are patients having the symptoms of constipation and/or gastrointestinal immotility and who have failed to obtain relief or ceased to obtain relief or a consistent degree of relief of their symptoms using a laxative or a stool softener, either alone or in combination, or who are otherwise resistant to laxatives and/or stool softeners. Such patients are said to be refractory to the conventional laxatives and/or stool softeners. The constipation and/or gastrointestinal immotility may be induced or a consequence of one or more diverse conditions including, but not limited to, a disease condition, a physical condition, a drug-induced condition, a physiological imbalance, stress, anxiety, and the like. The conditions inducing constipation and/or gastrointestinal immotility may be acute conditions or chronic conditions.

The subjects can be treated with a combination of (R)-7,8-saturated-4,5-epoxy-morphinanium, or the 3-O-protected-(R)-7,8-saturated-4,5-epoxy-morphinanium intermediate thereof, or prodrug thereof, and a laxative and/or a stool softener (and optionally, an opioid). In these circumstances the (R)-7,8-saturated-4,5-epoxy-morphinanium or the intermediate thereof and the other therapeutic agent(s) may be administered close enough in time such that the subject experiences the effects of the various agents as desired, which typically is at the same time. In some embodiments the (R)-7,8-saturated-4,5-epoxy-morphinanium analogs or the intermediate thereof will be delivered first in time, in some embodiments second in time, and still in some embodiments at the same time. As discussed in greater detail herein, the invention contemplates pharmaceutical preparations where the (R)-7,8-saturated-4,5-epoxy-morphinanium, or intermediate thereof, or prodrug thereof, is administered in a formulation including the (R)-7,8-saturated-4,5-epoxy-morphinanium or the intermediate thereof (or prodrug thereof) and one or both of a laxative and a stool softener (and, optionally, an opioid). These formulations may be parenteral or oral, such as the ones described in U.S. Ser. No. 10/821,809. Included are solid, semisolid, liquid, controlled release, lyophilized and other such formulations.

In an embodiment, the administered amount of (R)-7,8-saturated-4,5-epoxy-morphinanium is sufficient to induce Taxation. This has particular application where the subject is a chronic opioid user. Chronic opioid use as used herein includes daily opioid treatment for a week or more or intermittent opioid use for at least two weeks. It has been reported that patients receiving opioids chronically become tolerant to opioids and need increasing doses. Thus, a patient receiving oral doses of opioids chronically could be receiving between 40 and 100 mg per day of a morphine-equivalent dose of opioid. Certain (R)-7,8-saturated-4,5-epoxy-morphinaniums may require a different dose, in patients that have become more tolerant to opioids and taken an increasing dose.

Patients using opioids chronically include late stage cancer patients, elderly patients with osteoarthritic changes, methadone maintenance patients, neuropathic pain and chronic back pain patients. Treatment of these patients is important from a quality of life standpoint, as well as to reduce complications arising from chronic constipation, such as hemorrhoids, appetite suppression, mucosal breakdown, sepsis, colon cancer risk, and myocardial infarction.

Patients receiving treatment using the compounds of the present invention may concurrently or sequentially be receiving opioids. The opioid can be any pharmaceutically acceptable opioid. Common opioids are those selected from the group consisting of alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and tramadol. The opioid also may be mixed together with the (R)-7,8-saturated-4,5-epoxy-morphinanium or intermediate thereof and provided in any of the forms described above in connection with (R)-7,8-saturated-4,5-epoxy-morphinanium or intermediate thereof. Optionally, a non-opioid analgesic/anti-pyretic such as acetaminophen may be administered with the opioid, in particular with oxycodone.

Dosage may be adjusted appropriately to achieve desired drug levels, local or systemic, depending on the mode of administration. For example, it is expected that the dosage for oral administration of the opioid antagonists in an enterically-coated formulation would be lower than in an immediate release oral formulation. In the event that the response in a patient is insufficient at such doses, even higher doses (or effectively higher dosage by a different, more localized delivery route) may be employed to the extent that the patient tolerance permits. Multiple doses per day are contemplated to achieve appropriate systemic levels of compounds. Appropriate systemic levels can be determined by, for example, measurement of the patient's peak or sustained plasma level of the drug. “Dose” and “dosage” are used interchangeably herein.

A variety of administration routes are available. The particular mode selected will depend, of course, upon the particular combination of drugs selected, the severity of the condition being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, transdermal, sublingual, intravenous infusion, pulmonary, intra-arterial, intra-adipose tissue, intra-lymphatic, intramuscular, intracavity, aerosol, aural (e.g., via eardrops), intranasal, inhalation, intra-articular, needleless injection, subcutaneous or intradermal (e.g., transdermal) delivery. For continuous infusion, a patient-controlled analgesia (PCA) device or an implantable drug delivery device may be employed. Oral, rectal, or topical administration may be important for prophylactic or long-term treatment. Preferred rectal modes of delivery include administration as a suppository or enema wash.

The pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

It should be understood that when referring to 7,8-saturated-4,5-epoxy-morphinaniums, (R)- and (S)-7,8-saturated-4,5-epoxy-morphinanium, and therapeutic agent(s) of the invention, it is meant to encompass salts of the same. Such salts are of a variety well known to those or ordinary skill in the art. When used in pharmaceutical preparations, the salts preferably are pharmaceutically-acceptable for use in humans. Bromide is an example of one such salt.

The pharmaceutical preparations of the present invention may include or be diluted into a pharmaceutically-acceptable carrier. The term “pharmaceutically-acceptable carrier” as used herein means one or more compatible solid or liquid fillers, diluents or encapsulating substances which are suitable for administration to a human or other mammal such as non-human primate, a dog, cat, horse, cow, sheep, pig, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Formulations may include a chelating agent, a buffering agent, an anti-oxidant and, optionally, an isotonicity agent, preferably pH adjusted, and a permeation enhancer. Examples of such formulations that are stable to autoclaving and long term storage are described in co-pending U.S. application Ser. No. 10/821,811, entitled “Pharmaceutical Formulation.”

Chelating agents include, for example, ethylenediaminetetraacetic acid (EDTA) and derivatives thereof, citric acid and derivatives thereof, niacinamide and derivatives thereof, sodium desoxycholate and derivatives thereof, and L-glutamic acid, N,N-diacetic acid and derivatives thereof. EDTA derivatives include dipotassium edetate, disodium adetate, calcium-disodium edetate, sodium edetate, trisodium edetate, and potassium edetate.

Buffering agents include those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, or combinations thereof.

Antioxidants include those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopheral and derivatives thereof, monothioglycerol, and sodium sulfite. The preferred antioxidant is monothioglycerol.

Isotonicity agents include those selected from the group consisting of sodium chloride, mannitol, lactose, dextrose, glycerol, and sorbitol.

Preservatives that can be used with the present compositions include benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

The compounds of the invention can be prepared in lyophilized compositions, preferably in the presence of a cryoprotecting agent such as mannitol, or lactose, sucrose, polyethylene glycol, and polyvinyl pyrrolidines. Cryoprotecting agents which result in a reconstitution pH of 6.0 or less are preferred. The invention therefore provides a lyophilized preparation of therapeutic agent(s) of the invention. The preparation can contain a cryoprotecting agent, such as mannitol or lactose, which is preferably neutral or acidic in water.

Oral, parenteral and suppository formulations of agents are well known and commercially available. The therapeutic agent(s) of the invention can be added to such well known formulations. It can be mixed together in solution or semi-solid solution in such formulations, can be provided in a suspension within such formulations or could be contained in particles within such formulations.

A product containing therapeutic agent(s) of the invention and, optionally, one or more other active agents can be configured as an oral dosage. The oral dosage may be a liquid, a semisolid or a solid. An opioid may optionally be included in the oral dosage. The oral dosage may be configured to release the therapeutic agent(s) of the invention before, after or simultaneously with the other agent (and/or the opioid). The oral dosage may be configured to have the therapeutic agent(s) of the invention and the other agents release completely in the stomach, release partially in the stomach and partially in the intestine, in the intestine, in the colon, partially in the stomach, or wholly in the colon. The oral dosage also may be configured whereby the release of the therapeutic agent(s) of the invention is confined to the stomach or intestine while the release of the other active agent is not so confined or is confined differently from the therapeutic agent(s) of the invention. For example, the therapeutic agent(s) of the invention may be an enterically coated core or pellets contained within a pill or capsule that releases the other agent first and releases the therapeutic agent(s) of the invention only after the therapeutic agent(s) of the invention passes through the stomach and into the intestine. The therapeutic agent(s) of the invention also can be in a sustained release material, whereby the therapeutic agent(s) of the invention is released throughout the gastrointestinal tract and the other agent is released on the same or a different schedule. The same objective for therapeutic agent(s) of the invention release can be achieved with immediate release of therapeutic agent(s) of the invention combined with enteric coated therapeutic agent(s) of the invention. In these instances, the other agent could be released immediately in the stomach, throughout the gastrointestinal tract or only in the intestine.

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

In the event that it is desirable to release the therapeutic agent(s) of the invention first, the therapeutic agent(s) of the invention could be coated on the surface of the controlled release formulation in any pharmaceutically acceptable carrier suitable for such coatings and for permitting the release of the therapeutic agent(s) of the invention, such as in a temperature sensitive pharmaceutically acceptable carrier used for controlled release routinely. Other coatings which dissolve when placed in the body are well known to those of ordinary skill in the art.

The therapeutic agent(s) of the invention also may be mixed throughout a controlled release formulation, whereby it is released before, after or simultaneously with another agent. The therapeutic agent(s) of the invention may be free, that is, solubilized within the material of the formulation. The therapeutic agent(s) of the invention also may be in the form of vesicles, such as wax coated micropellets dispersed throughout the material of the formulation. The coated pellets can be fashioned to immediately release the therapeutic agent(s) of the invention based on temperature, pH or the like. The pellets also can be configured so as to delay the release of the therapeutic agent(s) of the invention, allowing the other agent a period of time to act before the therapeutic agent(s) of the invention exerts its effects. The therapeutic agent(s) of the invention pellets also can be configured to release the therapeutic agent(s) of the invention in virtually any sustained release pattern, including patterns exhibiting first order release kinetics or sigmoidal order release kinetics using materials of the prior art and well known to those of ordinary skill in the art.

The therapeutic agent(s) of the invention also can be contained within a core within the controlled release formulation. The core may have any one or any combination of the properties described above in connection with the pellets. The therapeutic agent(s) of the invention may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The therapeutic agent(s) of the invention, may be provided in particles. Particles as used herein means nano or microparticles (or in some instances larger) which can consist in whole or in part of the therapeutic agent(s) of the inventions or the other agents as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” These formulations may be for any mode of administration.

Delivery systems specific for the gastrointestinal tract are roughly divided into three types: the first is a delayed release system designed to release a drug in response to, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestinal tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36° C. to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale (Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site, physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the trade name EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT L, particularly L-30D and EUDRAGIT L 100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics. In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allows optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. The rate of water influx and drug efflux can be controlled by the composition and size of the orifice of the device. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesultonate), semipenneable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose dicylate, cellulose acetate succinate, cellulose propionate succinate, cellulose acetate octanoate, cellulose valerate palmitate, cellulose acetate heptanate, cellulose acetaldehyde dimethyl acetal, cellulose acetate ethylcarbamate, cellulose acetate methylcarbamate, cellulose dimethylaminoacetate and ethylcellulose.

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above. A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation of the compounds of the invention as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include Endragit L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% aid a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, sold under the trademark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

A product containing therapeutic agent(s) of the invention can be configured as a suppository. The therapeutic agent(s) of the invention can be placed anywhere within or on the suppository to favorably affect the relative release of the therapeutic agent(s). The nature of the release can be zero order, first order, or sigmoidal, as desired.

Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention includes, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface-active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition, may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic agents may be incorporated into the nasal delivery system or any other delivery system described herein.

A composition formulated for topical administration may be liquid or semi-solid (including, for example, a gel, lotion, emulsion, cream, ointment, spray or aerosol) or may be provided in combination with a “finite” carrier, for example, a non-spreading material that retains its form, including, for example, a patch, bioadhesive, dressing or bandage. It may be aqueous or non-aqueous; it may be formulated as a solution, emulsion, dispersion, a suspension or any other mixture.

Various modes of administration include topical application to the skin, eyes or mucosa. Thus, typical vehicles are those suitable for pharmaceutical or cosmetic application to body surfaces. The compositions provided herein may be applied topically or locally to various areas in the body of a patient. As noted above, topical application is intended to refer to application to the tissue of an accessible body surface, such as, for example, the skin (the outer integument or covering) and the mucosa (the mucous-producing, secreting and/or containing surfaces). Exemplary mucosal surfaces include the mucosal surfaces of the eyes, mouth (such as the lips, tongue, gums, cheeks, sublingual and roof of the mouth), larynx, esophagus, bronchial, nasal passages, vagina and rectum/anus; in some embodiments, preferably the mouth, larynx, esophagus, vagina and rectum/anus; in other embodiments, preferably the eyes, larynx, esophagus, bronchial, nasal passages, and vagina and rectum/anus. As noted above, local application herein refers to application to a discrete internal area of the body, such as, for example, a joint, soft tissue area (such as muscle, tendon, ligaments, intraocular or other fleshy internal areas), or other internal area of the body. Thus, as used herein, local application refers to applications to discrete areas of the body.

With respect to topical and/or local administration of the present compositions, desirable efficacy may involve, for example, penetration of therapeutic agent(s) of the invention into the skin and/or tissue to substantially reach a hyperalgesic site to provide desirable anti-hyperalgesic pain relief. The efficacy of the present compositions may be about the same as that achieved, for example, with central opiate analgesics. But, as discussed in detail herein, the efficacy achieved with therapeutic agent(s) of the invention is preferably obtained without the undesirable effects that are typically associated with central opiates including, for example, respiratory depression, sedation, and addiction, as it is believed that therapeutic agent(s) of the invention does not cross the blood brain barrier.

Also in certain embodiments, including embodiments that involve aqueous vehicles, the compositions may also contain a glycol, that is, a compound containing two or more hydroxy groups. A glycol which is particularly preferred for use in the compositions is propylene glycol. In these embodiments, the glycol is preferably included in the compositions in a concentration of from greater than 0 to about 5 wt. %, based on the total weight of the composition. More preferably, the compositions contain from about 0.1 to less than about 5 wt. % of a glycol, with from about 0.5 to about 2 wt. % being even more preferred. Still more preferably, the compositions contain about 1 wt. % of a glycol.

For local internal administration, such as intra-articular administration, the compositions are preferably formulated as a solution or a suspension in an aqueous-based medium, such as isotonically buffered saline or are combined with a biocompatible support or bioadhesive intended for internal administration.

Lotions, which, for example, may be in the form of a suspension, dispersion or emulsion, contain an effective concentration of one or more of the compounds. The effective concentration is preferably to deliver an effective amount, typically at a concentration of between about 0.1-50% [by weight] or more of one or more of the compounds provided herein. The lotions also contain [by weight] from 1% to 50% of an emollient and the balance water, a suitable buffer, and other agents as described above. Any emollients known to those of skill in the art as suitable for application to human skin may be used. These include, but are not limited to, the following: (a) Hydrocarbon oils and waxes, including mineral oil, petrolatum, paraffin, ceresin, ozokerite, microcrystalline wax, polyethylene, and perhydrosqualene. b) Silicone oils, including dimethylpolysiloxanes, methylphenylpolysiloxanes, water-soluble and alcohol-soluble silicone-glycol copolymers. (c) Triglyceride fats and oils, including those derived from vegetable, animal and marine sources. Examples include, but are not limited to, castor oil, safflower oil, cotton seed oil, corn oil, olive oil, cod liver oil, almond oil, avocado oil, palm oil, sesame oil, and soybean oil. (d) Acetoglyceride esters, such as acetylated monoglycerides. (e) Ethoxylated glycerides, such as ethoxylated glyceryl monstearate. (f) Alkyl esters of fatty acids having 10 to 20 carbon atoms. Methyl, isopropyl and butyl esters of fatty acids are useful herein. Examples include, but are not limited to, hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. (g) Alkenyl esters of fatty acids having 10 to 20 carbon atoms. Examples thereof include, but are not limited to, oleyl myristate, oleyl stearate, and oleyl oleate. (h) Fatty acids having 9 to 22 carbon atoms. Suitable examples include, but are not limited to, pelargonic, lauric, myristic, palmitic, stearic, isostearic, hydroxystearic, oleic, linoleic, ricinoleic, arachidonic, behenic, and erucic acids. (i) Fatty alcohols having 10 to 22 carbon atoms, such as, but not limited to, lauryl, myristyl, cetyl, hexadecyl, stearyl, isostearyl, hydroxystearyl, oleyl, ricinoleyl, behenyl, erucyl, and 2-octyl dodecyl alcohols. (j) Fatty alcohol ethers, including, but not limited to ethoxylated fatty alcohols of 10 to 20 carbon atoms, such as, but are not limited to, the lauryl, cetyl, stearyl, isostearyl, oleyl, and cholesterol alcohols having attached thereto from 1 to 50 ethylene oxide groups or 1 to 50 propylene oxide groups or mixtures thereof. (k) Ether-esters, such as fatty acid esters of ethoxylated fatty alcohols. (l) Lanolin and derivatives, including, but not limited to, lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases. (m) polyhydric alcohols and polyether derivatives, including, but not limited to, propylene glycol, dipropylene glycol, polypropylene glycol [M.W. 2000-4000], polyoxyethylene polyoxypropylene glycols, polyoxypropylene polyoxyethylene glycols, glycerol, ethoxylated glycerol, propoxylated glycerol, sorbitol, ethoxylated sorbitol, hydroxypropyl sorbitol, polyethylene glycol [M.W. 200-6000], methoxy polyethylene glycols 350, 550, 750, 2000, 5000, poly(ethylene oxide) homopolymers [M.W. 100,000-5,000,000], polyalkylene glycols and derivatives, hexylene glycol (2-methyl-2,4-pentanediol), 1,3-butylene glycol, 1,2,6,-hexanetriol, ethohexadiol USP (2-ethyl-1,3-hexanediol), C.sub.15-C.sub.18 vicinal glycol and polyoxypropylene derivatives of trimethylolpropane. (n) polyhydric alcohol esters, including, but not limited to, ethylene glycol mono- and di-fatty acid esters, diethylene glycol mono- and di-fatty acid esters, polyethylene glycol [M.W. 200-6000], mono- and di-fatty esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. (o) Wax esters, including, but not limited to, beeswax, spermaceti, myristyl myristate, and stearyl stearate and beeswax derivatives, including, but not limited to, polyoxyethylene sorbitol beeswax, which are reaction products of beeswax with ethoxylated sorbitol of varying ethylene oxide content that form a mixture of ether-esters. (p) Vegetable waxes, including, but not limited to, carnauba and candelilla waxes. (q) phospholipids, such as lecithin and derivatives. (r) Sterols, including, but not limited to, cholesterol and cholesterol fatty acid esters. (s) Amides, such as fatty acid amides, ethoxylated fatty acid amides, and solid fatty acid alkanolamides.

The lotions further preferably contain [by weight] from 1% to 10%, more preferably from 2% to 5%, of an emulsifier. The emulsifiers can be nonionic, anionic or cationic. Examples of satisfactory nonionic emulsifiers include, but are not limited to, fatty alcohols having 10 to 20 carbon atoms, fatty alcohols having 10 to 20 carbon atoms condensed with 2 to 20 moles of ethylene oxide or propylene oxide, alkyl phenols with 6 to 12 carbon atoms in the alkyl chain condensed with 2 to 20 moles of ethylene oxide, mono- and di-fatty acid esters of ethylene oxide, mono- and di-fatty acid esters of ethylene glycol where the fatty acid moiety contains from 10 to 20 carbon atoms, diethylene glycol, polyethylene glycols of molecular weight 200 to 6000, propylene glycols of molecular weight 200 to 3000, glycerol, sorbitol, sorbitan, polyoxyethylene sorbitol, polyoxyethylene sorbitan and hydrophilic wax esters. Suitable anionic emulsifiers include, but are not limited to, the fatty acid soaps, e.g., sodium, potassium and triethanolamine soaps, where the fatty acid moiety contains from 10 to 20 carbon atoms. Other suitable anionic emulsifiers include, but are not limited to, the alkali metal, ammonium or substituted ammonium alkyl sulfates, alkyl arylsulfonates, and alkyl ethoxy ether sulfonates having 10 to 30 carbon atoms in the alkyl moiety. The alkyl ethoxy ether sulfonates contain from 1 to 50 ethylene oxide units. Among satisfactory cationic emulsifiers are quaternary ammonium, morpholinium and pyridinium compounds. Certain of the emollients described in preceding paragraphs also have emulsifying properties. When a lotion is formulated containing such an emollient, an additional emulsifier is not needed, though it can be included in the composition.

The balance of the lotion is water or a C₂ or C₃ alcohol, or a mixture of water and the alcohol. The lotions are formulated by simply admixing all of the components together. Preferably the compound, such as loperamide, is dissolved, suspended or otherwise uniformly dispersed in the mixture.

Other conventional components of such lotions may be included. One such additive is a thickening agent at a level from 1% to 10% by weight of the composition. Examples of suitable thickening agents include, but are not limited to: cross-linked carboxypolymethylene polymers, ethyl cellulose, polyethylene glycols, gum tragacanth, gum kharaya, xanthan gums and bentonite, hydroxyethyl cellulose, and hydroxypropyl cellulose.

Creams can be formulated to contain a concentration effective to deliver an effective amount of therapeutic agent(s) of the invention to the treated tissue, typically at between about 0.1%, preferably at greater than 1% up to and greater than 50%, preferably between about 3% and 50%, more preferably between about 5% and 15% therapeutic agent(s) of the invention. The creams also contain from 5% to 50%, preferably from 10% to 25%, of an emollient and the remainder is water or other suitable non-toxic carrier, such as an isotonic buffer. The emollients, as described above for the lotions, can also be used in the cream compositions. The cream may also contain a suitable emulsifier, as described above. The emulsifier is included in the composition at a level from 3%, to 50%, preferably from 5% to 20%.

These compositions that are formulated as solutions or suspensions may be applied to the skin, or, may be formulated as an aerosol or foam and applied to the skin as a spray-on. The aerosol compositions typically contain [by weight] from 25% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as understood in the art in a quantity and under a pressure suitable to expel the contents of the container.

Suitably prepared solutions and suspensions may also be topically applied to the eyes and mucosa. Solutions, particularly those intended for ophthalmic use, may be formulated as 0.01%-10% isotonic solutions, pH about 5-7, with appropriate salts, and preferably containing one or more of the compounds herein at a concentration of about 0.1%, preferably greater than 1%, up to 50% or more. Suitable ophthalmic solutions are known [see, e.g., U.S. Pat. No. 5,116,868, which describes typical compositions of ophthalmic irrigation solutions and solutions for topical application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D.L.-sodium, .β.-hydroxybutyrate and 5-5.5 mM glucose.

Gel compositions can be formulated by simply admixing a suitable thickening agent to the previously described solution or suspension compositions. Examples of suitable thickening agents have been previously described with respect to the lotions.

The gelled compositions contain an effective amount of therapeutic agent(s) of the invention, typically at a concentration of between about 0.1-50% by weight or more of one or more of the compounds provided herein from 5% to 75%, preferably from 10% to 50%, of an organic solvent as previously described; from 0.5% to 20%, preferably from 1% to 10% of the thickening agent; the balance being water or other aqueous or non-aqueous carrier, such as, for example, an organic liquid, or a mixture of carriers.

The formulations can be constructed and arranged to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic settings, the therapeutic agent(s) of the invention will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will be obtained when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

To improve oral bioavailability of the compounds of the present invention, excipients may be used that increase intestinal membrane permeability (Aungst, B. J. J Pharmaceutical Science Vol 89, Issue 4, pp. 429-442, 2000). Permeation enhancers may include surfactants, fatty acids, medium chain glycerides, steroidal detergents, acyl carnitine and alkanoylcholines, N-acetylated alpha-amino acids and N-acetylated non-alpha-amino acids, and chitosans, and other mucoadhesive polyers. Specific examples include: cholate, glycocholate, glycosursodeoxycholate, ethylenediaminetetraacetic acid, hydroxypropyl-beta-cyclodextrin, hydroxypropyl-gamma-cylcodextrin, gamma-cylcodextrin, tetradecyl-beta-D-maltose, octylglucoside, citric acid, glycyrrhetinic acid, and Tween-80® (Shah, R. B. et al J Pharm. Sci April 93(4):1070-82, 2004).

Included within embodiments, is a kit which includes a container containing an opioid formulation and a container containing a (R)-7,8-saturated-4,5-epoxy-morphinanium formulation. In one (R)-7,8-saturated-4,5-epoxy-morphinanium formulation, the formulation is tablets which contain pellets, some of which are enterically coated with pH sensitive material and some of which are constructed and arranged to release the (R)-7,8-saturated-4,5-epoxy-morphinanium immediately in the stomach. The kit may also includes instructions for administering the tablets to a subject who is constipated or who has symptoms of constipation or gastrointestinal immotility. The instructions may include indicia, for example writing, indicating that the (R)-7,8-saturated-4,5-epoxy-morphinanium is pure (R)-7,8-saturated-4,5-epoxy-morphinanium free of its (S)-7,8-saturated-4,5-epoxy-morphinanium stereoisomer. The kit may include a pharmaceutical preparation vial, and a pharmaceutical preparation diluents vial The diluents vial may, for example, contain diluents such as physiological saline for diluting what could be a concentrated solution or lyophilized powder of (R)-7,8-saturated-4,5-epoxy-morphinanium. The instructions can include instructions for mixing a particular amount of the diluents with a particular amount of the concentrated pharmaceutical preparation, whereby a final formulation for injection or infusion is prepared. The instructions may include instructions for treating a patient with an effective amount of (R)-7,8-saturated-4,5-epoxy-morphinanium. It also will be understood that the containers containing the preparations, whether the container is a bottle, a vial with a septum, an ampoule with a septum, an infusion bag, and the like, can contain additional indicia such as conventional markings which change color when the preparation has been autoclaved or otherwise sterilized.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

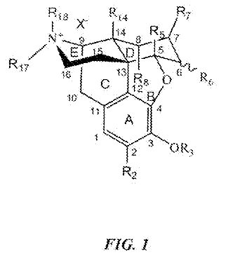

FIG. 1 provides one of the potential structures of a 7,8-saturated-4,5-epoxy-morphinanium embodiment of the present invention.

### EXAMPLE 1

### (R)-17-Allyl-17-cyclopropylmethyl-4,5α-epoxy-3,14-dihydroxy-6-oxomorphinanium Iodide

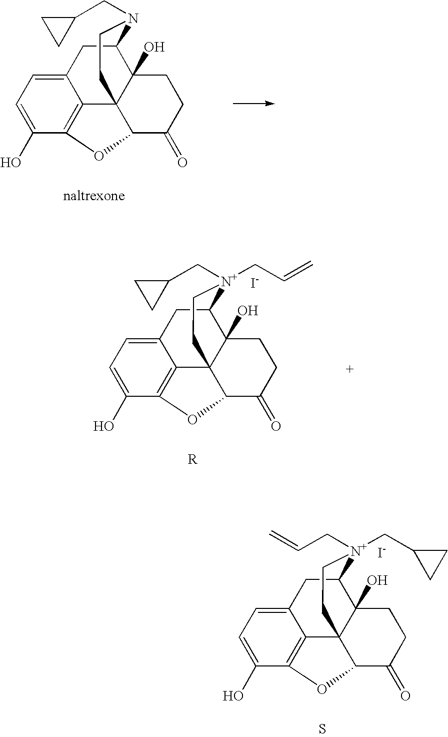

Synthetic Procedure. Naltrexone (2.0 g, 5.86 mmol) was dissolved in DMF (10 mL, anhydrous) under nitrogen. Allyl iodide (0.5 mL, 5.18 mmol) was added. The mixture was stirred at room temperature for 4 days. DMF was removed. The residue was stirred with 50 mL of water for 10 min. The aqueous solution was separated from the solid precipitates and washed with dichloromethane (50 mL). It was lyophilized to give a hygroscopic solid (1.2 g). 0.2 g of this solid was dissolved in water (30 mL). The pH of the water solution was adjusted to 10 by Na₂CO₃. This solution was washed with dichloromethane (2×20 mL) and lyophilized to give a yellow solid. This solid was purified by a reverse phase column (4 g, C18) to 28 mg of a solid which was later identified as a mixture of F 27-R and F27-S. The remaining of the above hygroscopic solid (˜1.0 g) was subjected to the same treatments to give another 81 mg solid as a mixture of (R) and S. This 81 mg solid was separated by semi-prep HPLC to give 55 mg (2%) of (R) and 9.5 mg (0.3%) of S.

R: ¹H NMR (300 MHz, D₂O) δ 6.83 (d, J=8.4 Hz, 1H), 6.77 (d, J=8.4 Hz, 1H), 6.14-6.04 (m, 1H), 5.73-5.67 (m, 1H), 5.13-5.04 (m, 1H), 5.04 (s, 1H), 4.97-4.89 (m, 1H), 3.72-3.58 (m, 3H), 3.17-2.83 (m, 5H), 2.30-2.25 (m, 1H), 2.16-2.09 (m, 1H), 1.88-1.78 (m, 1H), 1.24-1.14 (m, 1H), 0.85-0.75 (m, 2H), 0.52-0.42 (m, 2H). MS [M⁺]: 382.2. HPLC purity: 99% (UV detection at 254 nm).

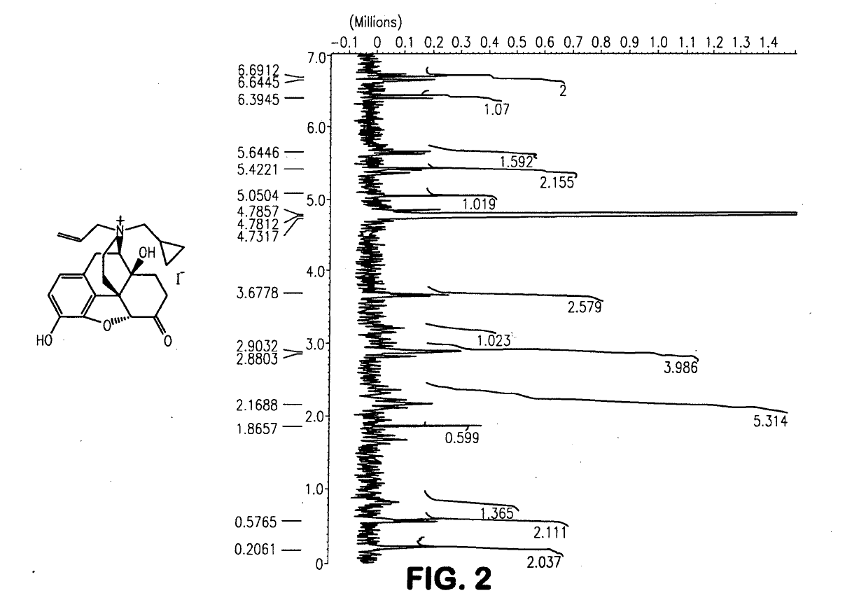

FIG. 2 provides a proton NMR spectrum of (S)-17-allyl-17-cyclopropylmethyl-4,5α-epoxy-3,14-dihydroxy-6-oxomprhinanium iodide.

S: ¹H NMR (300 MHz, D₂O) δ 6.67 (d, J=8.4 Hz, 1H), 6.39 (d, J=8.4 Hz, 1H), 6.64 (m, 1H), 5.5.42 (m, 2H), 5.05 (s, 1H), 4.8 (m, 2H), 3.68 (m, 2H), 3.17 (m, 1H), 2.90 (m, 4H), 2.40 (m, 1H), 2.16 (m, 4H), 1.70 (m, 1H), 0.83 (m, 1H), 0.58 (m, 2H), 0.21 (m, 2H). MS [M⁺]: 382.2. HPLC purity: 99% (UV detection at 254 nm).

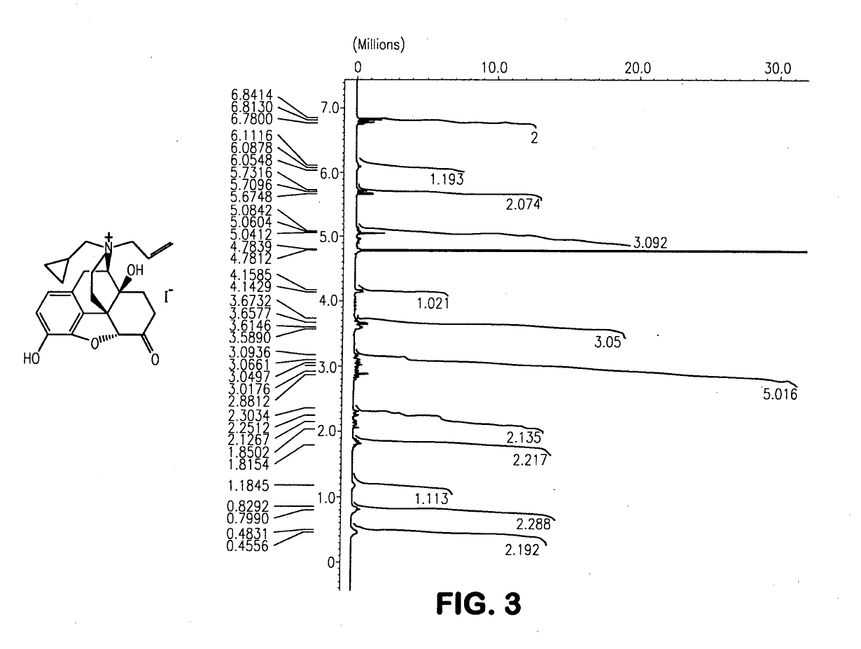

FIG. 3 provides a proton NMR spectrum of (R)-17-allyl-17-cyclopropylmethyl-4,5α-epoxy-3,14-dihydroxy-6-oxomorphinanium iodide.

### EXAMPLE 2

### (R)-17-cyclopropylmethyl-4,5α-epoxy-3,14-dihydroxy-17-methyl-6-methylenemorphinanium Iodide (F25)

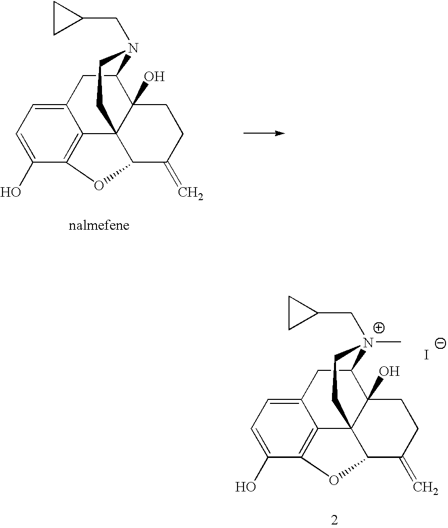

Synthetic Procedure. To a solution of Nalmefene (500 mg, 1 eq.) in NMP (2 mL) was added methyl iodide (1 mL, 10 eq.) and warmed to 55° C. The reaction mixture was kept stirring at this temperature for 80 hours. The crude reaction mixture was purified by passing through a reverse phase C-18 column using water-methanol solvent mixture as eluent (gradient elution) to afford the title compound 2 as a white powder (60

¹HNMR (300 MHz, D₂O) δ 6.80 (d, J=8.25 Hz, 1H), 6.73 (d, J=8.25 Hz, 1H), 5.28 (s, 1H), 5.18 (s, 1H), 4.94 (s, 1H), 3.96 (m, 2H), 3.63 (s, 3.2H), 3.57 (s, 0.63H), 3.28 (m, 1H), 3.18 (m, 1H), 3.03 (m, 1H), 2.71 (m, 2H), 2.56 (m, 1H), 2.18 (m, 1H), 1.75 (m, 2H), 1.41 (td, 1H, J=3.84, 13.4 Hz), 1.20 (m, 1H), 0.80 (m, 2H), 0.56 (m, 1H), 0.37 (m, 1H). MS [M⁺]: 354.28. HPLC purity: 93.5% (UV detection at 254 nm)

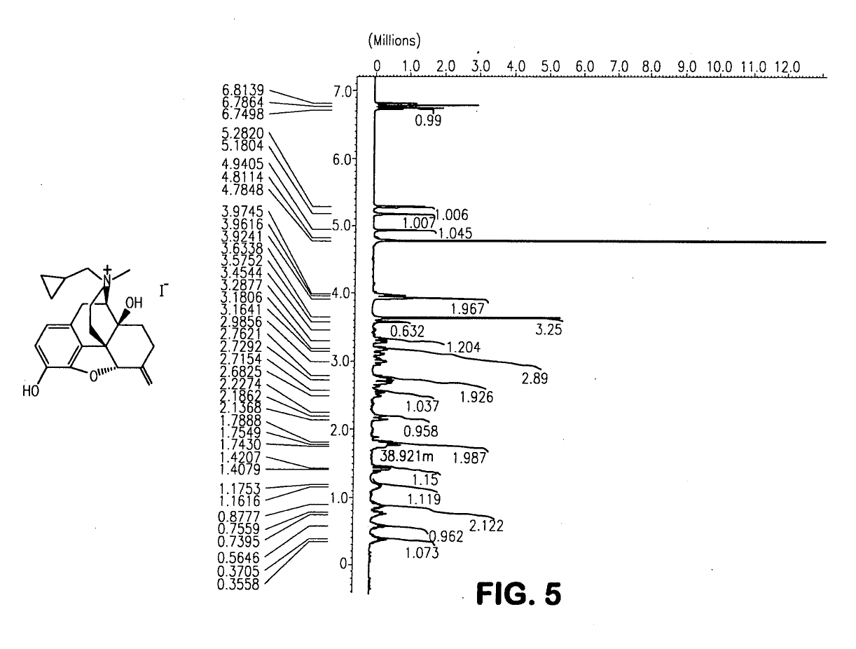

FIG. 5 provides a proton NMR spectrum of (R)-17-cyclopropylmethyl-4,5α-epoxy-3,14-dihydroxy-17-methyl-6-methylenemorphinanium iodide

### EXAMPLE 3

### (R)-17-Cyclopropylmethyl-4,5α-epoxy-3,14-dihydroxy-17-methyl-6β-hydroxy-8-propoxy-morphinanium trifluoroacetate

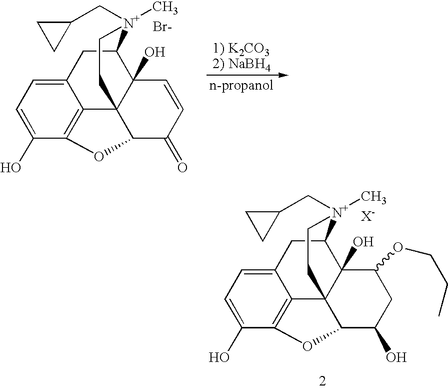

Synthetic Procedure. A mixture of delta 7-methylnaltrexone bromide (120 mg, 0.4 mmol) and powdered potassium carbonate (1 mg, 0.07 mmol) in n-propanol was heated on a steam bath and then allowed to cool to room temperature overnight. HPLC analysis showed 13% of 8-propoxy-N-methyl naltrexone intermediate. DBU (50 mg) was added and the reaction stirred and additional 4 hrs HPLC analysis showed 12% product. Additional potassium carbonate (100 mg, 0.72 mmol) was added an the reaction continued overnight at room temperature. HPLC analysis showed that the amount of intermediate had reduced to 9%. The reaction was charged with sodium borohydride (4 mg, 0.1 mmol) and stirred at room temperature overnight. In the morning another portion of sodium borohydride (4 mg, 0.1 mmol) was added and reaction was warmed in hot tap water and stirred overnight again. The solvent was removed in vacuo and the residue dissolved in 5 ml of 0.1% trifluoroacetic acid in 95:5 water:methanol and loaded onto a reversed phase C18 column (Biotage, 40 M) eluted with a linear gradient of 95:5 to 35:65 water:methanol with 0.1% trifluoroacetic acid. The product containing fractions were combined and the solvent was removed in vacuo to give 21.4 mg of product 2 (15% yield, 96% purity by HPLC, 90:6 ratio of isomers 60:6a).

¹H NMR (300 MHz, CD₃OD) δ 6.77 (s, 2H), 4.86 (s, 1H), 4.42 (d, 1H), 4.04 (br d, 1H), 3.9 (dd, 1H), 3.7 (s, 3H), 3.6-3.2 (m, 4H), 3.2-2.7 (m, 5H), 2.1-1.5 (m, 6H), 1.25 (m, 1H), 0.95 (t, J=7.3, 3H), 0.85 (m, 1H), 0.65 (m, 1H), 0.48 (m, 1H). MS [M⁺]: 417.2. HPLC purity: 95.2% (UV detection at 280 nm).

### EXAMPLE 4

### (R)-17-cyclobutylmethyl-4,5α-epoxy-3,14-dihydroxy-17-methyl-6-oxomorphinanium Iodide (B5)

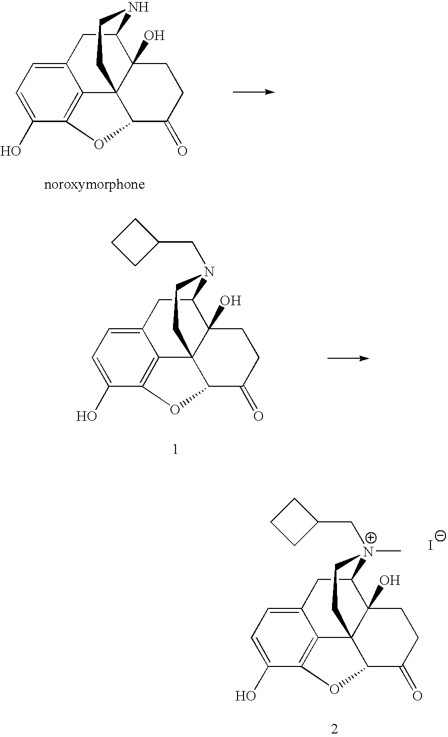

Synthetic Procedure.

(i) 17-cyclobutylmethyl-4,5α-epoxy-3,14-dihydroxy-17-methyl-6-oxomorphin. To a solution of noroxymorphone (500 mg, 1 eq.) in DMF (5 mL) was added sodium bicarbonate (160 mg, 1.1 eq.) and cyclobutyl methyl bromide ((215 μL, 1.1 eq.). The reaction mixture was stirred overnight at 90° C. The reaction mixture was cooled down to room temperature and diluted with chloroform (20 mL) and washed with brine. The aqueous washings were extracted (3×50 mL) with chloroform and the organics were pooled. The combined chloroform extracts were dried over anhydrous MgSO₄ and concentrated. The product was purified by silica column chromatography (10 g SiO₂) using dichloromethane-methanol (98:2) as eluent to afford 178 mg (47%) of compound 1.

(ii) (R)-17-cyclobutylmethyl-4,5α-epoxy-3,14-dihydroxy-17-methyl-6-oxomorphinanium Iodide (B5). To a solution of compound 1 (419 mg, 1 eq.) in 2 ml, of NMP was added methyl iodide (735 μL 10 eq.) and stirred at room temperature for 80 hours. The crude reaction mixture was partitioned between dichloromethane and sodium bicarbonate solution (pH >10). The aqueous phase was lyophilized to get light brown solid which was purified by passing through a reverse phase C-18 column using water-methanol solvent mixture as eluent (gradient elution) to afford the title compound 2 as a white powder (14 mg).

¹HNMR (300 MHz, D₂O) δ 6.81 (d, J=8.25 Hz, 1H), 6.75 (d, J=8.25 Hz, 1H), 5.01 (s, 1H), 3.93 (d, J=4.02, 1H), 3.69 (m, 1H), 3.53 (s, 3H), 3.38 (m, 3H), 3.02 (m, 5H), 2.19 (m, 7H), 1.

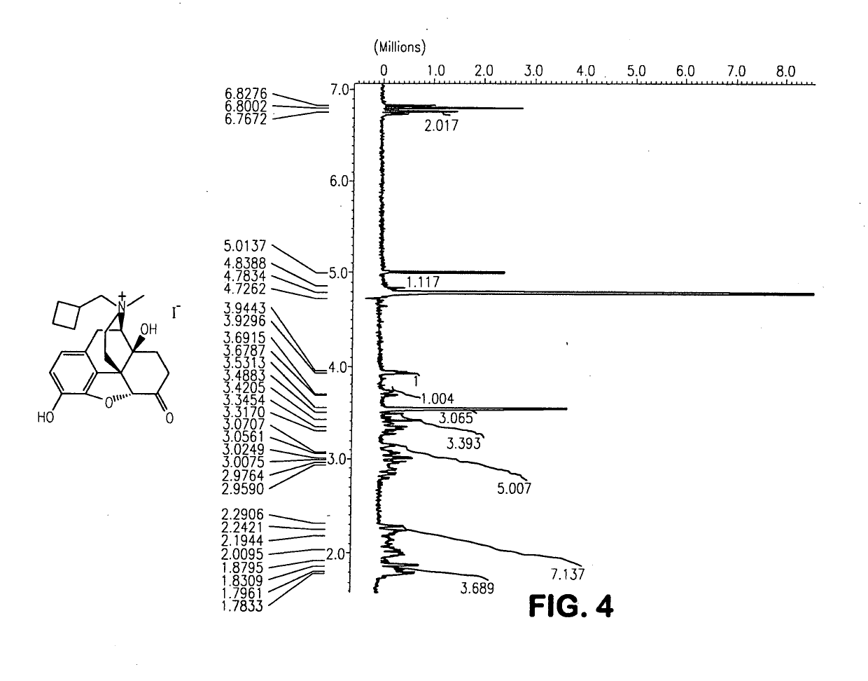

FIG. 4 provides a proton NMR spectrum of (R)-17-cyclobutylmethyl-4,5α-epoxy-3,14-dihydroxy-17-methyl-6-oxomorphinanium iodide; 79 (m, 3H). MS [M⁺]: 370.8. HPLC purity: 98% (UV detection at 254 nm).

### EXAMPLE 5

### (R)-17-cyclopentylmethyl-4,5α-epoxy-3,14-dihydroxy-17-methyl-6-methylenemorphinanium Iodide (B2)

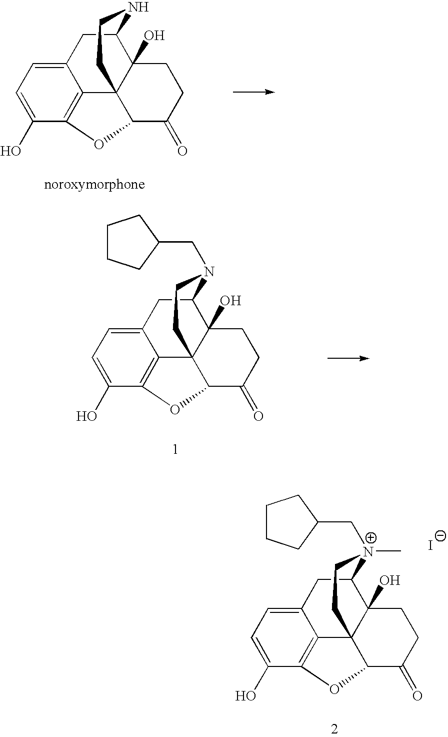

Synthetic Procedure.

(i) 17-cyclopentylmethyl-4,5α-epoxy-3,14-dihydroxy-17-methyl-6-oxomorphin (1). To a solution of noroxymorphone (502 mg, 1 eq.) in DMF (5 mL) was added sodium bicarbonate (160 mg, 1.1 eq.) and cyclopentylmethyl iodide (251 μL, 1.1 eq.). The reaction mixture was stirred overnight at 90° C. The reaction mixture was cooled down to room temperature and diluted with chloroform (20 mL) and washed with brine. The aqueous washings were extracted (3×50 mL) with chloroform and the organics were pooled. The combined chloroform extracts were dried over anhydrous Mg₂SO₄ and concentrated. The product was purified by silica column chromatography (10 g SiO₂) using dichloromethane-methanol (98:2) as eluent to afford 322 mg (50%) of compound 1.

(ii) (R)-17-cyclopentylmethyl-4,5α-epoxy-3,14-dihydroxy-17-methyl-6-oxomorphinanium Iodide, O-5281. To a solution of compound 1 (322 mg, 1 eq.) in 2 mL of NMP was added methyl iodide (542 μL, 10 eq.) and stirred at room temperature for 80 hours. The crude reaction mixture was partitioned between dichloromethane and sodium bicarbonate solution (pH >10). The aqueous phase was lyophilized to get an off-white solid which was purified by passing through a reverse phase C-18 column using water-methanol as eluent (gradient) to afford the title compound 2 as a light yellow solid which was further purified by semi-prep HPLC using water/methanol (70/30) with 0.1% TFA to afford 9 mg of title compound 2 as a white solid.

¹HNMR (300 MI z, D₂O) δ 6.80 (d, J=8.25 Hz, 1H), 6.75 (d, J=8.25 Hz, 1H), 5.00 (s, 1H), 3.98 (d, J=4.11, 1H), 3.83 (m, 1H), 3.61 (s, 3H), 3.48 (m, 1H), 3.37 (m, 1H), 3.05 (m, 6H), 2.27 (m, 1H), 2.02 (m, 3H), 1.76 (m, 6H), 1.25 (m, 2H). MS [M⁺]: 384.3. HPLC purity: 100% (UV detection at 254 nm).

### EXAMPLE 6

### Pharmacology

### Effects of (R)-17-cyclopropylmethyl-4,5α-epoxy-3,14-dihydroxy-17-methyl-6β-8-propoxy-morphinanium trifluoroacetate (“(R)-CPM”) (71) and (R)-17-cyclopentylmethyl-4,5α-epoxy-3,14-dihydroxy-17-methyl-6-oxomorphinanium iodide (“(R)-CPTM”) Evaluated for Agonist and Antagonist Activities at the μ-Opioid Receptors in the Guinea Pig Ileum

Agonist/antagonist activity at the μ-opioid receptor was determined using the well known guinea pig ileum test. Briefly, an a section of ileum section was placed in a stabilizing solution in a tensed state. Transducers were used to measure changes in tension upon electrical stimulation to the tissue before and after challenge with a potential agonist/antagonist Using a control, constriction inhibition, and constriction inhibition cancellation, may be measured.

Evaluation of agonist activity

| | Control | | | | | | | | | | + |
|---|---|---|---|---|---|---|---|---|---|---|---|
| | response to | Responses to increasing concentrations of the compounds | | | | | | | | | Naloxone |
| | DAMGO | (M) | | | | | | | | | (1.0E−07 M) |
| Compounds | (1.O−07 M) | 1.0E−08 | 3.0E−08 | 1.0E−07 | 3.0E−07 | 1.0E−06 | 3.0E−06 | 1.0E−05 | 3.0E−05 | 1.0E−04 | 1.0E−04 |
| (R)-CPM | 100 | 0 | 0 | 0 | 0 | 0 | 0 | 0 | 5 | 22 | 17 |
| (R)-CPTM | 100 | 0 | 0 | 0 | 0 | 0 | 0 | 0 | 12 | 18 | 18 |

| | | 1.0E−09 | 1.0E−08 | 1.0E−07 | 1.0E−06 | |
|---|---|---|---|---|---|---|
| DAMGO | 100 | 9 | 57 | 96 | 103 | 4 |

Evaluation of antagonist acitivity

| | | Responses to DAMGO (1.0E−07 M) in the presence of | | | | | | | | |
|---|---|---|---|---|---|---|---|---|---|---|
| | Control response | increasing concentrations of the compounds | | | | | | | | |
| | to DAMGO | (M) | | | | | | | | |
| Compounds | (1.0E−07 M) | 1.0E−08 | 3.0E−08 | 1.0E−07 | 3.0E−07 | 1.0E−06 | 3.0E−06 | 1.0E−05 | 3.0E−05 | 1.0E−04 |
| (R)-CPM | 100 | 100 | 100 | 90 | 69 | 32 | 11 | 5 | 12 | 22 |
| (R)-CPTM | 100 | 100 | 96 | 84 | 63 | 21 | −1 | 28 | 34 | 40 |

| | | |
|---|---|---|
| 5.0E−09 | 20E−08 | 1.0E−07 |

| | | | | |
|---|---|---|---|---|
| naloxone | 100 | 85 | 51 | −6 |

The results are expressed as a percent of the control response to DAMGO (decrease in which contract amplitude)

The disclosures of all patents, patent applications and scientific publications cited or referenced herein are incorporated by reference where appropriate for teachings of additional or alternative details, features, and/or technical background, including U.S. patent application Ser. Nos. 11/441,395 entitled “Synthesis of (R)—N-Methylnaltrexone” and 11/441,452 entitled “(S)—N-Methylnaltrexone” filed May 25, 2006. In case of conflict between documents incorporated by reference and the instant application the instant application will control.

Having thus described several embodiments of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### Statement Regarding Embodiments

While the invention has been described with respect to embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

What is claimed is:

1. An isolated compound of the (R) configuration with respect to the nitrogen of Formula I(c): 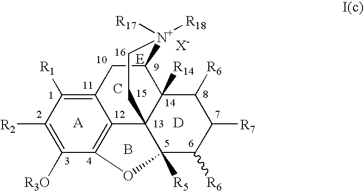
or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof,
or R₁ and R₂ can also be combined to form a C₃-C₆ carbocycle fused ring which may be substituted according to R₁₉, a benzo fused ring, or a 5-6 membered heteroaryl fused ring;
R₃ is H, silyl, CO₂R₁₉, SO₂R₁₉, B(OR₂₆)₂;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
C₁-C₃ acyl
R₅ is H, OH, OR₂₆,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, OH, OR₂₆, =(R₁₉)(R_{19′}), =(hetero cycle substituted with 0-3R₂₀), =(C₃-C₇ cycle substituted with 0-3R₂₀);
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
amine, amide, sulfonamide, or ester;
R₇ and R₈ are independently H, hydrocarbyl, cyclohydrocarbyl, hetero cycle with 0-3R₂₀, alkylaryl with 0-3R₂₀, arylakly with 0-3 R₂₀, or substituted moieties thereof, or 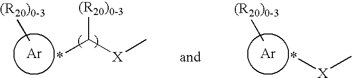
where, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19′}), COO;
R₇ and R₈ are combined to form a carbocycle fused ring which may be substituted according to R₁₉, a benzo fused ring, 5-, 6-, or a 5-6 membered aryl or heteroaryl with 0-3R₂₀;
R₁₄ is H, OH, OR₂₆, NR₂₂R₂₃SR₂₅, S(═O)R₂₅, SO₂R₂₅, hetero cycle with 0-3R₂₀, alkylaryl with 0-3R₂₀, arylalkyl with 0-3R₂₀, 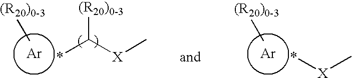
wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl, hetero cycle with 0-3R₂₀, alkylaryl with 0-3R₂₀, arylalkyl with 0-3_{R20}, 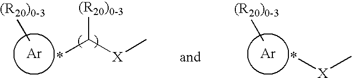
wherein, X is bond, ═O, O, S, N(R₁₉), SO, SO₂, SO₂N(R₁₉), CON(R₁₉), N(R₁₉)CON(R_{19′}), N(R₁₉)C(═NR_{19′})N(R_{19″}), COO;
R₁₉ is at each occurrence is independently selected from: H, C₁-C₆ alkyl, CF₃, OR₂₄, Cl, F, Br, I, ═O, CN, NO₂, NR₂₂R₂₃ aryl substituted with 0-3R₂₀;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂,
NR₂₂R₂₃, acetyl, OR₂₅, XR₂₅,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂,
NR₂₂R₂₃, CF₃, acetyl, OR₂₅, XR₂₅,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, hetero aryl, hetero cycle, alkylaryl, and arylalkyl;
(C₁-C₆ alkyl)-C(═O)—, and (C₁-C₆ alkyl)-S(═O)₂—;
R₂₃, at each occurrence, is independently selected from: H, (C₁-C₆)alkyl, benzyl, phenethyl, C₆-C₁₀ aryl hetero aryl, hetero cycle, alkylaryl, haloalkyl, arylalkyl
(C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅ is alkyl, aryl, or arylalkyl;
R₂₆ is at each occurrence is independently selected from:
H, C₁-C₆ alkyl, CF₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

2. An isolated compound of the (R) configuration with respect to the nitrogen of Formula I: 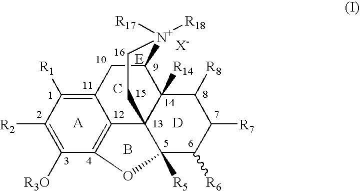
or a pharmaceutically acceptable salt form or prodrug form thereof, wherein:
R₁ and R₂ are independently H, OH, OR₂₆, halide, silyl; hydrocarbyl, cyclohydrocarbyl, or substituted moieties thereof; or R₁ and R₂ can also be combined to form a C₃-C₆ carbocycle fused ring which may be substituted according to R₁₉, a benzo fused ring, or a 5-6 membered heteroaryl fused ring;
R₃ is H, silyl;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
C₁-C₃ acyl
R₅ is H, OH, OR₂₆,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, OH, OR₂₆;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
amine, amide, sulfonamide, or ester;
R₇ and R₈ are independently H, hydrocarbyl, cyclohydrocarbyl, or substituted moieties thereof; or R₇ and R₈ are combined to form a carbocycle fused ring which may be substituted according to R₁₉, a benzo fused ring, or a 5-6 membered heteroaryl fused ring;
R₁₄ is H, OH, OR₂₆, NR₂₂R₂₃SR₂₅, S(═O)R₂₅, SO₂R₂₅;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ can be combined with R₁₇ or R₁₈ depending on its configuration with respect to quaternary nitrogen to form an O-fused ring, or a C₃-C₆ carbocycle fused ring;
R₁₇ and R₁₈ are C₁-C₆ hydrocarbyls which may be substituted, wherein if R₁₈ is methyl, R₁₇ is not allyl;
R₁₉ is at each occurrence is independently selected from:
H, C₁-C₆ alkyl, CF₃, OR₂₄, Cl, F, Br, I, ═O, CN, NO₂, NR₂₂R₂₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂,
NR₂₂R₂₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂,
NR₂₂R₂₃, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl,
homopiperidinyl, thiomorpholinyl, piperizinyl, and morpholinyl;
R₂₂, at each occurrence, is independently selected from H, C₁-C₆ alkyl, benzyl, phenethyl,
(C₁-C₆ alkyl)-C(═O)—, and (C₁-C₆ alkyl)-S(═O)₂—;
R₂₃, at each occurrence, is independently selected from:
H, (C₁-C₆)alkyl, benzyl, phenethyl,
(C₁-C₆ alkyl)-C(═O)—, and (C₁-C₆ alkyl)-S(═O)₂—;
or R₂₃ can be combined with R₂₂ to form a 5-, 6-, 5-7-membered cycle with 0-3R₂₀;
R₂₄, at each occurrence, is independently selected from H, phenyl, benzyl, (C₁-C₆) alkyl, haloalkyl, and (C₂-C₆) alkoxyalkyl;
R₂₅ is alkyl, aryl, XR₂₄, haloalkyl, or arylalkyl;
R₂₆ is at each occurrence is independently selected from:
H, C₁-C₆ alkyl, CF₃;
C₃-C₁₀ carbocycle substituted with 0-3 R₂₁;
aryl substituted with 0-3 R₂1; or
5 to 10 membered heterocycle containing 1 to 4 heteroatoms selected from nitrogen, oxygen, and sulphur, wherein said 5 to 10 membered heterocycle is substituted with 0-3 R₂₁; and
X⁻ is an anion.

3. The compound of Formula (I) according to claim 2, or a pharmaceutically acceptable salt form or prodrug form thereof, wherein the anion is a halide, sulfate, phosphate, nitrate, or anionic-charged organic species.

4. The compound of Formula (I) according to claim 3, or a pharmaceutically acceptable salt form or prodrug form thereof, wherein the anion is a halide.

5. The compound of Formula (I) according to claim 4, or a pharmaceutically acceptable salt form or prodrug form thereof wherein the halide is bromide or iodide.

6. The compound of Formula (I) according to claim 2, or a pharmaceutically acceptable salt form or prodrug form thereof, having at least 90% purity.

7. The compound of Formula (I) according to claim 2, or a pharmaceutically acceptable salt form or prodrug form thereof, having at least 95% purity.

8. The compound of Formula (I) according to claim 2, or a pharmaceutically acceptable salt form or prodrug form thereof, comprising a crystalline form.

9. The compound of Formula (I) according to claim 4, or a pharmaceutically acceptable salt form or prodrug form thereof, comprising a crystalline form.

10. The compound of Formula (I) according to claim 2, or a pharmaceutically acceptable salt form or prodrug form thereof, wherein the R-configuration is 95% pure with respect to the quaternary nitrogen

11. The compound of Formula (I) according to claim 2, or a pharmaceutically acceptable salt form or prodrug form thereof, wherein the R-configuration is 98% pure with respect to the quaternary nitrogen.

12. The compound of Formula (I) according to claim 2, or a pharmaceutically acceptable salt form or prodrug form thereof, wherein the R-configuration is 99.5% pure with respect to the quaternary nitrogen.

13. The compound of Formula (I) according to claim 2, or a pharmaceutically acceptable salt form or prodrug form thereof, wherein the R-configuration is 99.8% pure with respect to the quaternary nitrogen.

14. A composition comprising the compound according claim 2 or a pharmaceutically acceptable salt form or prodrug form thereof, wherein the R-configuration is about 90% pure with respect to the quaternary nitrogen.

15. The composition of claim 14, wherein the composition is a solution.

16. The composition of claim 14, wherein the composition is a solid.

17. A pharmaceutical composition comprising a therapeutically effective amount of the compound of claim 2, and a pharmaceutically acceptable carrier.

18. The pharmaceutical composition of claim 17 wherein the composition is an oral formulation.

19. The pharmaceutical composition of claim 17 wherein the composition is in a controlled release or sustained release formulation.

20. The pharmaceutical composition of claim 17, wherein the composition is a topical formulation.

21. The pharmaceutical composition of claim 17, wherein the composition is lyophilized.

22. The pharmaceutical composition of claim 17, wherein the composition is a suppository.

23. An inhaler containing the pharmaceutical composition of claim 17.

24. A nasal spray device containing the pharmaceutical composition of claim 17.

25. The pharmaceutical composition of claim 17, wherein the compound of claim 2 is in the R configuration with respect to the nitrogen and the composition contains HPLC detectable S configuration counterpart stereoisomer at a detection limit of 0.02% and a quantitation limit of 0.05%.

26. The pharmaceutical composition of claim 17, wherein the composition is free of HPLC detectable S configuration counterpart at a detection level of 0.02% and at a quantitation level of 0.05%.

27. An isolated 3-O-protected compound salt of claim 2 wherein the protecting group is selected from the group consisting of: isobutyryl, 2-methyl butyryl, tertbutyl carbonyl, silyl ethers, 2-tetrahydropyranyl ethers, and alkyl carbonates.

28. The composition of claim 17, further comprising a therapeutic agent other than (S) counterpart stereoisomer.

29. The composition of claim 28, wherein the therapeutic agent is an opioid agonist.

30. The pharmaceutical composition of claim 28, wherein the opioid is selected from the group consisting of alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucuronide, nalbuphine, nalorphine, opium, oxycodone, oxymorphone, pentazocine, propiram, propoxyphene, remifentanyl, sufentanil, tilidine, trimebutine, tramadol, and combinations thereof.

31. The pharmaceutical composition of claim 14, further comprising at least one pharmaceutical agent that is not an opioid or an opioid antagonist.

32. The pharmaceutical composition of claim 31, wherein at least one pharmaceutical agent is non-opioid/anti-pyretic, an antiviral agent, an anti-infective agent, an anticancer agent, an antispasmodic agent, an anti-muscarinic agent, an anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, a herbal medicine, an anti-emetic agent, an anti-diarrheal agent, a laxative, a stool softener, a fiber or a hematopoietic stimulating agent.

33. The composition of claim 32, wherein the anti-inflammatory agent is selected from the group consisting of non-steroidal anti-inflammatory drugs (NSAIDS), tumor necrosis factor inhibitors, basiliximab, daclizumab, infliximab, mycophenolate, mofetil, azothioprine, tacrolimus, steroids, sulfasalazine, olsalazine, mesalamine, and combinations thereof.

34. A pharmaceutical composition comprising the compound of claim 2 and a pharmaceutically acceptable carrier.

35. The pharmaceutical composition of claim 34 enterically coated for oral administration.

36. The pharmaceutical composition of claim 34 in a lyophilized formulation.

37. The pharmaceutical composition of claim 34 in a sustained release formulation or immediate release formulation.

38. The pharmaceutical composition of claim 37, further comprising an opioid.

39. The pharmaceutical composition of claim 38, wherein the opioid is selected from the group consisting of alfentanil, anileridine, asimodiline, bremazocine, burprenorphine, butorphanol, codeine, dezocine, diacetylmorphine (heroin), dihydrocodeine, diphenyloxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, tramadol, and combinations thereof.

40. The pharmaceutical composition of claim 39, further comprising at least one pharmaceutical agent that is not an opioid or an opioid antagonist.

41. The pharmaceutical composition of claim 40, wherein at least one pharmaceutical agent is an antiviral agent, an anti-infective agent, an anticancer agent, an antispasmodic agent, a non-opioid analgesic/anti-pyretic, an anti-muscarinic agent, an anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, a herbal medicine, an anti-emetic agent, an anti-diarrheal agent, a laxative, a stool softener, a fiber or a hematopoietic stimulating agent.

42. The composition of claim 41, wherein the anti-inflammatory agent is selected from the group consisting of non-steroidal anti-inflammatory drugs (NSAIDS), tumor necrosis factor inhibitors, basiliximab, daclizumab, infliximab, mycophenolate, mofetil, azothioprine, tacrolimus, steroids, sulfasalazine, olsalazine, mesalamine, and combinations thereof.

43. A method for treating or preventing opioid-induced side effects comprising administering to a patient in need of such treatment the composition of claim 34 in an amount effective to treat or prevent the side effect.

44. A method for preventing or treating opioid-induced side effect in a patient chronically administered opioids, the method comprising administering a composition of claim 34 in an amount sufficient to prevent or treat the side effect in the patient.

45. A method of claim 44, wherein the side effect is selected from a group consisting of constipation, immune suppression, inhibition of gastrointestinal motility, inhibition of gastric emptying, nausea, emesis, incomplete evacuation, bloating, abdominal distension, increased gastroesophageal reflux, hypotension, bradycardia, gastrointestinal dysfunction, pruritus, dysphoria, and urinary retention.

46. A method for treating a patient receiving an opioid for pain resulting from surgery comprising administering to the patient a composition 34 of claim in an amount effective to promote gastrointestinal motility, gastric emptying or relief of constipation.

47. A method for treating or preventing endogenous opioid-induced dysfunction, comprising administering to a patient in need of such treatment the composition of claim 34 in an effective amount to treat the endogenous opioid-induced dysfunction.

48. The method of claim 27, wherein the gastrointestinal dysfunction is selected from a group consisting of inhibition of gastrointestinal motility, constipation and post-operative bowel dysfunction, obesity, hypertension, and addiction.

49. A method for preventing or treating idiopathic constipation comprising administering to a patient a composition of claim 34 in an amount effective to prevent or treat the idiopathic constipation.

50. A method for treating irritable bowel syndrome comprising administering to a patient in need of such treatment the composition of claim 34 in an amount effective to ameliorate at least one symptom of the irritable bowel syndrome.

51. The method of claim 50, further comprising administration of at least one irritable bowel syndrome therapeutic agent to the patient.

52. The method of claim 51, wherein the irritable bowel syndrome therapeutic is selected from the groups consisting of an antispasmodic agent, an anti-muscarinic agent, a non-steroidal or steroidal anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, an herbal medicine, an anti-diarrheal agent and combinations thereof.

53. A method for inducing laxation in a patient in need of Taxation comprising administering to a patient in need of such treatment the composition of claim 34 in an amount effective to induce laxation.

54. A method for preventing or treating post-operative bowel dysfunction comprising administering to a patient in need of such prevention or treatment the composition claim 34 in an amount effective to prevent or ameliorate at least one symptom of post-operative bowel dysfunction.

55. The method of claim 54 wherein, the post-operative bowel dysfunction is delayed gastric emptying or inhibition of gastrointestinal motility.

56. A method for treating or preventing opioid-induced side effects comprising administering to a patient in need of such treatment the compound of claim 2 in an amount effective to treat or prevent the side effect.

57. The method according to claim 56, wherein the patient is receiving opioids acutely or chronically.

58. A method of 56, wherein the side effect is selected from a group consisting of constipation, immune suppression, inhibition of gastrointestinal motility, inhibition of gastric emptying, nausea, emesis, incomplete evacuation, bloating, abdominal distension, increased gastroesophageal reflux, hypotension, bradycardia, gastrointestinal dysfunction, pruritus, dysphoria, and urinary retention.

59. The method of claim 58, wherein the opioid-induced side effect is constipation.

60. The method of claim 58, wherein the opioid-induced side effect is inhibition of gastrointestinal motility or inhibition of gastric emptying.

61. The method of claim 58, wherein the opioid-induced side effect is nausea or emesis.

62. The method of claim 58, wherein the opioid-induced side effect is pruritus.

63. The method of claim 58, wherein the opioid-induced side effect is dysphoria.

64. The method of claim 58, wherein the opioid-induced side effect is urinary retention.

65. A method for treating a patient receiving an opioid for pain resulting from surgery comprising administering to the patient a compound of claim 2 in an amount effective to promote gastrointestinal motility, gastric emptying or relief of constipation.

66. A method for treating or preventing endogenous opioid-induced gastrointestinal dysfunction, comprising administering to a patient in need of such treatment the compound of claim 2 in an effective amount to treat the endogenous opioid-induced gastrointestinal dysfunction.

67. The method of claim 66, wherein the gastrointestinal dysfunction is selected from a group consisting of inhibition of gastrointestinal motility, constipation and post-operative bowel dysfunction.

68. A method for preventing or treating idiopathic constipation comprising administering to a patient a compound of claim 2 in an amount effective to prevent or treat the idiopathic constipation.

69. A method for treating irritable bowel syndrome comprising administering to a patient in need of such treatment a compound of claim 2 in an amount effective to ameliorate at least one symptom of the irritable bowel syndrome.

70. The method of claim 69, further comprising administration of at least one irritable bowel syndrome therapeutic agent to the patient.

71. The method of claim 70, wherein the irritable bowel syndrome therapeutic is selected from the groups consisting of an antispasmodic agent, an anti-muscarinic agent, a non-steroidal or steroidal anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, an herbal medicine, an anti-diarrheal agent and combinations thereof.

72. An isolated compound of the (R)-stereoisomer of the formula Ia: 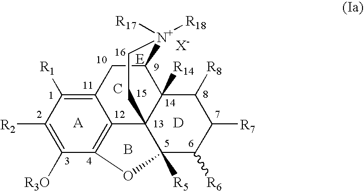
wherein
R₁₇ and R₁₈ are selected alternatively with respect to one another from (a) or (b):
(a) unsubstituted or non-halogen substituted: C₄-C₈ (cycloalkyl)alkyl or (cycloalkenyl)alkyl, (cycloheteryl)alkyl, (cycloaryl)alkyl; C₄-C₆ (cycloalkyl)alkyl or (cycloalkenyl)alkyl, (cycloheteryl)alkyl, (cycloaryl)alkyl
(b) substituted or unsubstituted linear or branched C₁-C₃ alkyl, C₂-C₃ alkenyl, or C₃ alkynyl;
wherein if (b) is selected as methyl, and R₆ is ═O, (a) is not unsubstituted (cyclopropyl)methyl;
R₆ is H, OH, ═O, ═CH₂, —N(CH₃)₂, or any cyclic ring, or forms a cyclic ring with R₇;
R₇ and R % are H or alkyl;
R₁₄ is H, OH, halide, arylamido, amino, N-alkyl, N-dialkyl, N-aryl, N-alkylaryl, N-cycloalkylalkyl, SCH₃, S(═O)CH₃, S(═O)₂CH₃, alkoxy, aryloxy, or aryl-alkoxy or forms a cyclic ring with R₁₇ or R₁₈;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

73. An isolated compound of the (R)-stereoisomer of the Formula Ib: 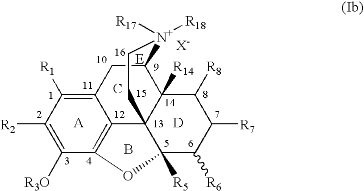
wherein
R₁₇ and R₁₈ are a substituted or unsubstituted C₁-C₆ hydrocarbyl, wherein when R₆ is selected as ═O, at least one of which is not methyl when the other is cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₈ are H or hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, SR₂₅, S(═O)R₂₅, SO₂R₂₅; alkoxy, aryloxy, or arylalkoxy, or forms a ring with R₁₇ or R₁₈;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.

74. A method of treatment comprising administering to a subject with a disorder characterized by unwanted migration or proliferation of endothelial cells an effective amount of a compound of claim 2.
